(19) United States
(12) Patent Application Publication
(10) Pub. No.: US 2003/0055014 A1
(43) Pub. Date: Mar. 20, 2003

(54) Inhibition of angiogenesis by nucleic acids

(76) Inventor: Robert L. Bratzler, Concord, MA (US)

Correspondence Address: Maria A. Trevisan, c/o Wolf, Greenfield & Sacks, P.C., Federal Reserve Plaza, 600 Atlantic Avenue, Boston, MA, 02210, US

(21) Appl. No.: 10/017,995

(22) Filed: Dec. 14, 2001

Related U.S. Application Data
(60) Provisional application No. 60/255,534, filed on Dec. 14, 2000.

(51) Int. Cl. A61K 48/00

(52) U.S. Cl. 514/44

(57) ABSTRACT

The invention relates to methods and products for inhibiting angiogenesis. At least one antiangiogenic nucleic acid molecule is administered to a subject to prevent or treat unwanted angiogenesis. Non-nucleic acid antiangiogenic agents also can be administered.

### RELATED APPLICATIONS

This application claims priority under 35 U.S.C. §119(e) from Provisional U.S. Patent Application Serial No. 60/255,534 filed on Dec. 14, 2000, entitled INHIBITION OF ANGIOGENESIS BY NUCLEIC ACIDS. The entire contents of the provisional application are hereby expressly incorporated by reference.

### BACKGROUND OF THE INVENTION

Blood vessels are the means by which oxygen and nutrients are supplied to living tissues and waste products are removed from living tissue. Angiogenesis refers to the process by which new blood vessels are formed. See, for example, the review by Folkman and Shing, J. Biol. Chem. 267(16):10931-10934, 1992. Thus, where appropriate, angiogenesis is a critical biological process. It is essential in reproduction, development and wound repair. However, inappropriate angiogenesis can have severe negative consequences. For example, it is only after many solid tumors are vascularized as a result of angiogenesis that the tumors have a sufficient supply of oxygen and nutrients that permit it to grow rapidly and metastasize. Because maintaining the rate of angiogenesis in its proper equilibrium is so critical to a range of functions, it must be carefully regulated in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to the formation of solid endothelial cell sprouts into the stromal space, then, vascular loops are formed and capillary tubes develop with formation of tight junctions and deposition of new basement membrane.

In adults, the proliferation rate of endothelial cells is typically low compared to other cell types in the body. The turnover time of these cells can exceed one thousand days. Physiological exceptions in which angiogenesis results in rapid proliferation typically occurs under tight regulation, such as found in the female reproduction system and during wound healing.

The rate of angiogenesis involves a change in the local equilibrium between positive and negative regulators of the growth of microvessels. The therapeutic implications of angiogenic growth factors were first described by Folkman and colleagues over two decades ago (Folkman, N. Engl. J. Med. 285:1182-1186, 1971). Abnormal angiogenesis occurs when the body loses at least some control of angiogenesis, resulting in either excessive or insufficient blood vessel growth. For instance, conditions such as ulcers, strokes, and heart attacks may result from the absence of angiogenesis normally required for natural healing. In contrast, excessive blood vessel proliferation can result in tumor growth, tumor spread, blindness, psoriasis and rheumatoid arthritis.

There are instances where a greater degree of angiogenesis is desirable, e.g., increasing blood circulation, wound healing, and ulcer healing. For example, recent investigations have established the feasibility of using recombinant angiogenic growth factors, such as fibroblast growth factor (FGF) family (Yanagisawa-Miwa et al., Science, 257:1401-1403, 1992; Baffour et al., J. Vasc. Surg. 16:181-91, 1992), endothelial cell growth factor (ECGF; Pu et al., J. Surg. Res. 54:575-83, 1993), and more recently, vascular endothelial growth factor (VEGF) to expedite and/or augment collateral artery development in animal models of myocardial and hindlimb ischemia (Takeshita et al., Circulation, 90:228-234, 1994; Takeshita et al., J. Clin. Invest. 93:662-70, 1994).

Conversely, there are instances where inhibition of angiogenesis is desirable. For example, many diseases are driven by persistent unregulated angiogenesis, also sometimes referred to as “neovascularization”. In arthritis, new capillary blood vessels invade the joint and destroy cartilage. In diabetes, new capillaries invade the vitreous of the eye, bleed, and cause blindness. Ocular neovascularization is the most common cause of blindness. Tumor growth and metastasis are angiogenesis-dependent. A tumor must continuously stimulate the growth of new capillary blood vessels for the tumor itself to grow.

The current approved treatment of these diseases is inadequate. Agents which prevent continued angiogenesis, such as drugs (e.g. TNP-470), monoclonal antibodies, antisense nucleic acids and proteins (e.g., angiostatin and endostatin) are currently being tested, but have not been approved. Although preliminary results with the antiangiogenic proteins are promising, they are relatively large in size and are difficult to use and produce. Moreover, proteins are subject to enzymatic degradation. Thus, new agents that inhibit angiogenesis are needed. New antiangiogenic agents that show improvement in size, ease of production, stability and/or potency would be desirable.

### SUMMARY OF THE INVENTION

It has now been discovered that nucleic acid molecules, including oligonucleotides, have intrinsic antiangiogenesis properties apart from the proteins such nucleic acids may encode.

According to one aspect of the invention, methods for inhibiting angiogenesis are provided. The methods include administering to a subject in need of such treatment at least one antiangiogenic nucleic acid molecule in an amount effective to inhibit angiogenesis in the subject. In some embodiments, two or more antiangiogenic nucleic acid molecules are administered. In other embodiments, non-nucleic acid antiangiogenic agents also are administered and agents that are effective against other aspects of an angiogenic condition (e.g., anticancer agents) can also be administered. In some embodiments, the angiogenesis is associated with a condition selected from the group consisting of rheumatoid arthritis, psoriasis, diabetic retinopathy, retinopathy of prematurity, macular degeneration, corneal graft rejection, neovascular glaucoma, retrolental fibroplasia, rubeosis, Osler-Webber Syndrome, myocardial angiogenesis, plaque neovascularization, telangiectasia, hemophiliac joints, angiofibroma, and wound granulation. In other embodiments, the angiogenesis is not associated with a cancer or tumor, but may be associated with an eye or ocular disorder such as those described herein. In still other embodiments, the angiogenesis is associated with embryo implantation. In certain embodiments, the angiogenesis is associated with conditions involving excessive or abnormal stimulation of endothelial cells such as but not limited to intestinal adhesions, atherosclerosis, scleroderma, and hypertrophic scars, i.e., keloids.

In other aspects of the invention, compositions are provided that include at least one antiangiogenic nucleic acid molecule, formulated in a pharmaceutically-acceptable carrier and in an effective amount for inhibiting angiogenesis. The compositions in certain embodiments include non-nucleic acid antiangiogenic agents and/or agents that are effective against other aspects of an angiogenic condition (e.g., anticancer agents).

According to still other aspects the invention, kits are provided that include a first container housing at least one antiangiogenic nucleic acid molecule and instructions for administering the antiangiogenic nucleic acid molecule to a subject having unwanted angiogenesis. In certain embodiments, a second container housing at least one non-nucleic acid antiangiogenic agent is also provided. In other embodiments of the foregoing kits, another container housing at least one anticancer agent is provided. In certain embodiments, the instructions relate to administering the antiangiogenic nucleic acid to a subject having a condition that is not cancer or a tumor, and examples of such conditions are listed throughout the specification.

A nucleic acid molecule is an element of each aspect of the invention. Preferred nucleic acid molecules include at least one sequence set forth as SEQ ID NOs: 1-1093. The nucleic acids useful according to the invention are synthetic or natural (isolated) nucleic acids. The nucleic acid may be administered alone or in conjunction with a pharmaceutically-acceptable carrier and optionally other therapeutic agents. In some embodiments the nucleic acid is a CpG nucleic acid, including those having an unmethylated CpG motif, a T-rich nucleic acid, or a poly G nucleic acid.

The nucleic acid in some embodiments has a nucleotide backbone which includes at least one backbone modification, such as a phosphorothioate modification or other phosphate modification. In some embodiments the modified backbone is a peptide modified oligonucleotide backbone. The nucleotide backbone may be chimeric, or the nucleotide backbone is entirely modified.

The nucleic acid can have any length greater than 6 nucleotides, but in some embodiments is between 8 and 100 nucleotide residues in length. In other embodiments the nucleic acid comprises at least 20 nucleotides, at least 24 nucleotides, at least 27, nucleotides, or at least 30 nucleotides. The nucleic acid may be single stranded or double stranded. In some embodiments the nucleic acid is isolated and in other embodiments the nucleic acid may be a synthetic nucleic acid. The antiangiogenic nucleic acids in some instances are not antisense molecules.

The CpG nucleic acid in one embodiment contains at least one unmethylated CpG dinucleotide having a sequence including at least the following formula: 5′ X₁ X₂CGX₃X₄ 3′ wherein C is unmethylated, wherein X₁, X₂, X₃, and X₄ are nucleotides. In one embodiment the 5′ X₁ X₂CGX₃ X₄ 3′ sequence of the CpG nucleic acid is a non-palindromic sequence, and in other embodiments it is a palindromic sequence.

In some embodiments X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA. In other embodiments X₁X₂ are GpA or GpT and X₃X₄ are TpT. In yet other embodiments X₁ or X₂ or both are purines and X₃ or X₄ or both are pyrimidines or X₁X₂ are GpA and X₃ or X₄ or both are pyrimidines. In one embodiment X₂ is a T and X₃ is a pyrimidine.

In other embodiments the CpG nucleic acid has a sequence selected from the group consisting of SEQ ID NO: 1, 3, 4, 14-16, 18-24, 28, 29, 33-46, 49, 50, 52-56, 58, 64-67, 69, 71, 72, 76-87, 90, 91, 93, 94, 96, 98, 102-124, 126-128, 131-133, 136-141, 146-150, 152-153, 155-171, 173-178, 180-186, 188-198, 201, 203-214, 216-220, 223, 224, 227-240, 242-256, 258, 260-265, 270-273, 275, 277-281, 286-287, 292, 295-296, 300, 302, 305-307, 309-312, 314-317, 320-327, 329, 335, 337-341, 343-352, 354, 357, 361-365, 367-369, 373-376, 378-385, 388-392, 394, 395, 399, 401-404, 406-426, 429-433, 434-437, 439, 441-443, 445, 447, 448, 450, 453-456, 460-464, 466-469, 472-475, 477, 478, 480, 483-485, 488, 489, 492, 493, 495-502, 504-505, 507-509, 511, 513-529, 532-541, 543-555, 564-566, 568-576, 578, 580, 599, 601-605, 607-611, 613-615, 617, 619-622, 625-646, 648-650, 653-664, 666-697, 699-706, 708, 709, 711-716, 718-732, 736, 737, 739-744, 746, 747, 749-761, 763, 766-767, 769, 772-779, 781-783, 785-786, 7900792, 798-799, 804-808, 810, 815, 817, 818, 820-832, 835-846, 849-850, 855-859, 862, 865, 872, 874-877, 879-881, 883-885, 888-904, and 909-913.

In some embodiments the T rich nucleic acid is a poly T nucleic acid comprising 5′ TTTT 3′. In yet other embodiments the poly T nucleic acid comprises 5′ X₁ X₂TTTTX₃ X₄ 3′ wherein X₁, X₂, X₃ and X₄ are nucleotides. In some embodiments X₁X₂ is TT and/or X₃X₄ is TT. In other embodiments X₁X₂ is selected from the group consisting of TA, TG, TC, AT, AA, AG, AC, CT, CC, CA, CG, GT, GG, GA, and GC; and/or X₃X₄ is selected from the group consisting of TA, TG, TC, AT, AA, AG, AC, CT, CC, CA, CG, GT, GG, GA, and GC.

The T rich nucleic acid may have only a single poly T motif or it may have a plurality of poly T nucleic acid motifs. In some embodiments the T rich nucleic acid comprises at least 2, at least 3, at least 4, at least 5, at least 6, at least 7, or at least 8 T motifs. In other embodiments it comprises at least 2, at least 3, at least 4, at least 5, at least 6, at least 7, or at least 8 CpG motifs. In some embodiments the plurality of CpG motifs and poly T motifs are interspersed.

In yet other embodiments at least one of the plurality of poly T motifs comprises at least 3, at least 4, at least 5, at least 6, at least 7, at least 8, or at least 9 contiguous T nucleotide residues. In other embodiments the plurality of poly T motifs is at least 3 motifs and wherein at least 3 motifs each comprises at least 3 contiguous T nucleotide residues or the plurality of poly T motifs is at least 4 motifs and wherein the at least 4 motifs each comprises at least 3 contiguous T nucleotide residues.

The T rich nucleic acid may include one or more CpG motifs. The motifs may be methylated or unmethylated. In other embodiments the T rich nucleic acid is free of one or more CpG dinucleotides.

In other embodiments the T rich nucleic acid has poly A, poly G, and/or poly C motifs. In other embodiments the T rich nucleic acid is free of two poly C sequences of at least 3 contiguous C nucleotide residues. Preferably the T rich nucleic acid is free of two poly A sequences of at least 3 contiguous A nucleotide residues. In other embodiments the T rich nucleic acid comprises a nucleotide composition of greater than 25% C or greater than 25% A. In yet other embodiments the T rich nucleic acid is free of poly-C sequences, poly G sequences or poly-A sequences.

In some cases the T rich nucleic acid may be free of poly T motifs, but rather, comprises a nucleotide composition of greater than 25% T. In other embodiments the T rich nucleic acid may have poly T motifs and also comprise a nucleotide composition of greater than 25% T. In some embodiments the T rich nucleic acid comprises a nucleotide composition of greater than 25% T, greater than 30% T, greater than 40% T, greater than 50% T, greater than 60% T, greater than 80% T, or greater than 90% T nucleotide residues. The T rich nucleic acid in some embodiments is selected from the group consisting of SEQ ID NOs: 59-63, 73-75, 142, 215, 226, 241, 267-269, 282, 301, 304, 330, 342, 358, 370-372, 393, 433, 471, 479, 486, 491, 497, 503, 556-558, 567, 694, 793-794, 797, 833, 852, 861, 867, 868, 882, 886, 905, 907, 908, and 910-913. In other embodiments the T rich nucleic acids are sequence selected from the group consisting of SEQ ID NOs: 64, 98, 112, 146, 185, 204, 208, 214, 224, 233, 244, 246, 247, 258, 262, 263, 265, 270-273, 300, 305, 316, 317, 343, 344, 350, 352, 354, 374, 376, 392, 407, 411-413, 429-432, 434, 435, 443, 474, 475, 498-501, 518, 687, 692, 693, 804, 862, 883, 884, 888, 890, and 891.

In some embodiments the poly G nucleic acid comprises: 5′ X₁X₂GGGX₃X₄ 3′ wherein X₁, X₂, X₃, and X₄ are nucleotides. In embodiments at least one of X₃ and X₄ are a G or both of X₃ and X₄ are a G. In other embodiments the poly G nucleic acid comprises the following formula: 5′ GGGNGGG 3′ wherein N represents between 0 and 20 nucleotides. In yet other embodiments the poly G nucleic acid comprises the following formula: 5′ GGGNGGGNGGG 3′ wherein N represents between 0 and 20 nucleotides. The poly G nucleic acid in some embodiments is selected from the group consisting of SEQ ID NOs.: 5, 6, 73, 215, 267-269, 276, 282, 288, 297-299, 355, 359, 386, 387, 444, 476, 531, 557-559, 733, 768, 795, 796, 914-925, 928-931, 933-936, and 938. In other embodiments the poly G nucleic acid includes a sequence selected from the group consisting of SEQ ID NOs; 67, 80-82, 141, 147, 148, 173, 178, 183, 185, 214, 224, 264, 265, 315, 329, 434, 435, 475, 519, 521-524, 526, 527, 535, 554, 565, 609, 628, 660, 661, 662, 725, 767, 825, 856, 857, 876, 892, 909, 926, 927, 932, and 937.

The poly G nucleic acid may include one or more CpG motifs or T-rich motifs. The CpG motifs may be methylated or unmethylated. In other embodiments the poly G nucleic acid is free of one or more CpG dinucleotides or poly-T motifs.

The nucleic acid molecules and optionally other agents may be administered by any route known in the art for delivering medicaments. The medicaments may be administered separately or together, in the same pharmaceutical formulation or separate formulations, by the same route or by different routes. In one embodiment the nucleic acid molecule(s) is administered on a routine schedule. In another embodiment the other agent(s) (e.g., antiangiogenesis agents, anticancer agents) is administered on a routine schedule.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a histogram showing the effect of a CpG nucleic acid on angiogenesis as measured by hemoglobin content.

The drawing is not required for enablement of the claimed invention.

### DETAILED DESCRIPTION OF THE INVENTION

The present invention includes compositions that include antiangiogenic nucleic acids and methods of using the antiangiogenic nucleic acids for the treatment of diseases that are mediated by angiogenesis. The invention includes antiangiogenic nucleic acids having various nucleotide sequences. The present invention comprises a method of treating undesired angiogenesis in a human or animal comprising the steps of the administering to the human or animal with the undesired angiogenesis a composition comprising an effective amount of, for example, an antiangiogenic nucleic acid.

As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” means a thin layer of flat epithelial cells that lines serous cavities, lymph vessels, and blood vessels. The term “endothelial inhibiting activity” means the capability of a molecule to inhibit angiogenesis in general and, for example, to inhibit the growth of bovine capillary endothelial cells in culture in the presence of fibroblast growth factor.

Antiangiogenic nucleic acids are effective in treating diseases or processes that are mediated by, or involve, angiogenesis. The present invention includes the method of treating an angiogenesis mediated disease with an effective amount of antiangiogenic nucleic acids. The angiogenesis mediated diseases include, but are not limited to, solid tumors; blood born tumors such as leukemias; tumor metastasis; benign tumors, for example hemangiomas, acoustic neuromas, neurofibromas, trachomas, and pyogenic granulomas; pre-malignant tumors; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation.

Antiangiogenic nucleic acids may be useful in the treatment of disease of excessive or abnormal stimulation of endothelial cells. These diseases include, but are not limited to, intestinal adhesions, atherosclerosis, scleroderma, and hypertrophic scars, i.e., keloids. Antiangiogenic nucleic acid can be used as a birth control agent by preventing vascularization required for embryo implantation.

Antiangiogenic nucleic acids may be useful in the treatment of conditions characterized by abnormal epithelial cell proliferation, such as proliferative dermatologic disorders. These include conditions such as keloids, seborrheic keratosis, papilloma virus infection (e.g. producing verruca vulbaris, verruca plantaris, verruca plana, condylomata, etc.) and eczema.

Antiangiogenic nucleic acids may be useful in the treatment of precancerous lesions such as epithelial precancerous lesions. An epithelial precancerous lesion is a lesion of epithelial cell origin that has a propensity to develop into a cancerous condition. An example is a precancerous skin lesion. Epithelial precancerous skin lesions also arise from other proliferative skin disorders such as hemangiomas, keloids, eczema and papilloma virus infections producing verruca vulbaris, verruca plantaris and verruca planar. The symptoms of the epithelial precancerous lesions include skin-colored or red-brown macule or papule with dry adherent scales. Actinic keratosis is the most common epithelial precancerous lesion among fair skinned individuals. It is usually present as lesions on the skin which may or may not be visually detectable. The size and shape of the lesions varies. It is a photosensitive disorder and may be aggravated by exposure to sunlight. Bowenoid actinic keratosis is another form of an epithelial precancerous lesion. In some cases, the lesions may develop into an invasive form of squamous cell carcinoma and may pose a significant threat of metastasis. Other types of epithelial precancerous lesions include hypertrophic actinic keratosis, arsenical keratosis, hydrocarbon keratosis, thermal keratosis, radiation keratosis, viral keratosis, Bowen's disease, erythroplaquia of queyrat, oral erythroplaquia, leukoplakia, and intraepidermal epithelialoma.

Antiangiogenic nucleic acids may be used in combination with other compositions and procedures for the treatment of diseases. For example, a tumor may be treated conventionally with surgery, radiation or chemotherapy combined with antiangiogenic nucleic acids and then antiangiogenic nucleic acids may be subsequently administered to the patient to extend the dormancy of micrometastases and to stabilize any residual primary tumor. In some instances it may be preferable to administer the antiangiogenic nucleic acids specifically to a site likely to harbor a metastatic lesion (that may or may not be clinically discernible at the time). A sustained release formulation implanted specifically at the site (or the tissue) where the metastatic lesion is likely to be would be suitable in these latter instances.

In some embodiments, the antiangiogenic nucleic acids of the invention do not interfere with specific receptor-ligand interactions at the cell surface of a cell, thereby causing the stimulation or inhibition of signaling through such receptors. These interactions include those involving heparin binding receptor, VEGF receptor, or EGF receptor.

In still other embodiments, the antiangiogenic nucleic acids are not antisense nucleic acids, meaning that they do not function by binding to complementary genomic DNA or RNA species within a cell and thereby inhibiting the function of said genomic DNA or RNA species. In important embodiments, the antiangiogenesis nucleic acid does not comprise a nucleic acid sequence that corresponds to a VEGF encoding sequence (or is complementary to a VEGF encoding sequence).

The effective dosage for inhibition of angiogenesis in vivo, which can be defined as inhibition of capillary endothelial cell proliferation and/or migration and/or blood vessel ingrowth, can be extrapolated from in vitro inhibition assays. In vitro assays have been developed to screen for inhibition of angiogenesis. Events that can be tested to assess angiogenesis inhibitors include proteolytic degradation of extracellular matrix and/or basement membrane, proliferation of endothelial cells, migration of endothelial cells, and capillary tube formation. The chick chorioallantoic membrane assay (CAM), described by Taylor and Folkman (Nature 297:307-312, 1982), can be used to determine whether the compound is capable of inhibiting neovascularization in vivo.

In some embodiments, the antiangiogenic nucleic acids are administered in doses, routes and schedules (and also in therapeutic cocktails) that would not result in the stimulation of an immune response.

The effective dosage is dependent not only on the sequence of the nucleic acid molecules used for inhibition of angiogenesis, but also on the method and means of delivery, which can be localized or systemic. For example, in some applications, as in the treatment of psoriasis or diabetic retinopathy, the inhibitor preferably is delivered in a topical or ophthalmic carrier. In other applications, as in the treatment of solid tumors, the inhibitor preferably is delivered by means of a biodegradable, polymeric implant.

An “antiangiogenic nucleic acid” as used herein is any nucleic acid containing an antiangiogenic motif or backbone that inhibits capillary endothelial cell proliferation and/or migration and/or blood vessel ingrowth.

The compounds useful according to the invention are nucleic acids. The nucleic acids may be double-stranded or single-stranded. Generally, double-stranded molecules may be more stable in vivo, while single-stranded molecules may have increased activity. The terms “nucleic acid” and “oligonucleotide” refer to multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)) or a modified base. As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules as used herein include vectors, e.g., plasmids, as well as oligonucleotides.

The terms “nucleic acid” and “oligonucleotide” also encompass nucleic acids or oligonucleotides with a covalently modified base and/or sugar. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide-nucleic acids (which have amino acid backbone with nucleic acid bases). In some embodiments the nucleic acids are homogeneous in backbone composition.

The substituted purines and pyrimidines of the nucleic acids include standard purines and pyrimidines such as cytosine as well as base analogs such as C-5 propyne substituted bases (Wagner et al., Nature Biotechnology 14:840-844, 1996). Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties.

The nucleic acid is a linked polymer of bases or nucleotides. As used herein with respect to linked units of a nucleic acid, “linked” or “linkage” means two entities are bound to one another by any physicochemical means. Any linkage known to those of ordinary skill in the art, covalent or non-covalent, is embraced. Such linkages are well known to those of ordinary skill in the art. Natural linkages, which are those ordinarily found in nature connecting the individual units of a nucleic acid, are most common. The individual units of a nucleic acid may be linked, however, by synthetic or modified linkages.

Whenever a nucleic acid is represented by a sequence of letters it will be understood that the nucleotides are in 5′→3′ order from left to right and that “A” denotes adenosine, “C” denotes cytosine, “G” denotes guanosine, “T” denotes thymidine, and “U” denotes uracil unless otherwise noted.

Nucleic acid molecules useful according to the invention can be obtained from natural nucleic acid sources (e.g. genomic nuclear or mitochondrial DNA or cDNA), or are synthetic (e.g. produced by oligonucleotide synthesis). Nucleic acids isolated from existing nucleic acid sources are referred to herein as native, natural, or isolated nucleic acids. The nucleic acids useful according to the invention may be isolated from any source, including eukaryotic sources, prokaryotic sources, nuclear DNA, mitochondrial DNA, etc. Thus, the term nucleic acid encompasses both synthetic and isolated nucleic acids.

The term “isolated” as used herein refers to a nucleic acid which is substantially free of or which is separated from components which it is normally associated with in nature e.g., nucleic acids, proteins, lipids, carbohydrates or in vivo systems to an extent practical and appropriate for its intended use. In particular, the nucleic acids are sufficiently pure and are sufficiently free from other biological constituents of host cells so as to be useful in, for example, producing pharmaceutical preparations. Because an isolated nucleic acid of the invention may be admixed with a pharmaceutically-acceptable carrier in a pharmaceutical preparation, the nucleic acid may comprise only a small percentage by weight of the preparation. The nucleic acid is nonetheless substantially pure in that it has been substantially separated from the substances with which it may be associated in living systems. The nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. After being administered to a subject the plasmid can be degraded into oligonucleotides. One skilled in the art can purify viral, bacterial, eukaryotic, etc. nucleic acids using standard techniques, such as those employing restriction enzymes, exonucleases or endonucleases.

For use in the instant invention, the nucleic acids can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et. al., Nucl. Acid. Res. 14:5399-5407, 1986, ; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

In some embodiments, the nucleic acids useful according to the invention may function as immunostimulatory nucleic acids. An immunostimulatory nucleic acid is any nucleic acid, as described herein, which is capable of modulating an immune response. A nucleic acid which modulates an immune response is one which produces any form of immune stimulation, including, but not limited to, induction of a cytokine, B cell activation, T cell activation, monocyte activation. Immunostimulatory nucleic acids include, but are not limited to, CpG nucleic acids, T-rich nucleic acids, poly G nucleic acids, and nucleic acids having phosphate modified backbones, such as phosphorothioate backbones.

A “CpG nucleic acid” or a “CpG antiangiogenic nucleic acid” as used herein is a nucleic acid containing at least one unmethylated CpG dinucleotide (cytosine-guanine dinucleotide sequence, i.e. “CpG DNA” or DNA containing a 5′ cytosine followed by 3′ guanosine and linked by a phosphate bond) and inhibits angiogenesis. The entire CpG nucleic acid can be unmethylated or portions may be unmethylated but at least the C of the 5′ CG 3′ must be unmethylated.

In one embodiment the invention provides a CpG nucleic acid represented by at least the formula:

5′N₁X₁CGX₂N₂3′

wherein X₁ and X₂ are nucleotides and N is any nucleotide and N₁ and N₂ are nucleic acid sequences composed of from about 0-25 N's each. In some embodiments X₁ is adenine, guanine, or thymine and/or X₂ is cytosine, adenine, or thymine. In other embodiments X₁ is cytosine and/or X₂ is guanine.

In other embodiments the CpG nucleic acid is represented by at least the formula:

5′N₁X₁X₂CGX₃X₄N₂3′

wherein X₁, X₂, X₃, and X₄ are nucleotides. In some embodiments, X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA; N is any nucleotide and N₁ and N₂ are nucleic acid sequences composed of from about 0-25 N's each. In some embodiments, X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines.

In another embodiment the CpG nucleic acid has the sequence

5′TCN₁TX₁X₂CGX₃X₄3′.

Examples of CpG nucleic acids according to the invention include but are not limited to those listed in Table 1, such as SEQ ID NOs: 1, 3, 4, 14-16, 18-24, 28, 29, 33-46, 49, 50, 52-56, 58, 64-67, 69, 71, 72, 76-87, 90, 91, 93, 94, 96, 98, 102-124, 126-128, 131-133, 136-141, 146-150, 152-153, 155-171, 173-178, 180-186, 188-198, 201, 203-214, 216-220, 223, 224, 227-240, 242-256, 258, 260-265, 270-273, 275, 277-281, 286-287, 292, 295-296, 300, 302, 305-307, 309-312, 314-317, 320-327, 329, 335, 337-341, 343-352, 354, 357, 361-365, 367-369, 373-376, 378-385, 388-392, 394, 395, 399, 401-404, 406-426, 429-433, 434-437, 439, 441-443, 445, 447, 448, 450, 453-456, 460-464, 466-469, 472-475, 477, 478, 480, 483-485, 488, 489, 492, 493, 495-502, 504-505, 507-509, 511, 513-529, 532-541, 543-555, 564-566, 568-576, 578, 580, 599, 601-605, 607-611, 613-615, 617, 619-622, 625-646, 648-650, 653-664, 666-697, 699-706, 708, 709, 711-716, 718-732, 736, 737, 739-744, 746, 747, 749-761, 763, 766-767, 769, 772-779, 781-783, 785-786, 7900792, 798-799, 804-808, 810, 815, 817, 818, 820-832, 835-846, 849-850, 855-859, 862, 865, 872, 874-877, 879-881, 883-885, 888-904, and 909-913.

A “T rich nucleic acid” or “T rich antiangiogenic nucleic acid” is a nucleic acid which includes at least one poly T sequence and/or which has a nucleotide composition of greater than 25% T nucleotide residues and which inhibits angiogenesis. A nucleic acid having a poly-T sequence includes at least four Ts in a row, such as 5′-TTTT-3′. Preferably the T rich nucleic acid includes more than one poly T sequence. In preferred embodiments the T rich nucleic acid may have 2, 3, 4, etc poly T sequences, such as SEQ ID NO:246 or SEQ ID NO:433. Other T rich nucleic acids have a nucleotide composition of greater than 25% T nucleotide residues, but do not necessarily include a poly T sequence. In these T rich nucleic acids the T nucleotide resides may be separated from one another by other types of nucleotide residues, i.e., G, C, and A. In some embodiments the T rich nucleic acids have a nucleotide composition of greater than 30%, 40%, 50%, 60%, 70%, 80%, 90%, and 99%, T nucleotide residues and every integer % in between. Preferably the T rich nucleic acids have at least one poly T sequence and a nucleotide composition of greater than 25% T nucleotide residues.

In one embodiment the T rich nucleic acid is represented by at least the formula:

5′X₁X₂TTTTX₃X₄3′

wherein X₁, X₂, X₃, and X₄ are nucleotides. In one embodiment X₁X₂ is TT and/or X₃X₄ is TT. In another embodiment X₁X₂ are any one of the following nucleotides TA, TG, TC, AT, AA, AG, AC, CT, CC, CA, CG, GT, GG, GA, and GC; and X₃X₄ are any one of the following nucleotides TA, TG, TC, AT, AA, AG, AC, CT, CC, CA, CG, GT, GG, GA, and GC.

In some embodiments it is preferred that the T-rich nucleic acid does not contain poly C (CCCC), poly A (AAAA), poly G (GGGG), CpG motifs, or multiple GGs. In other embodiments the T-rich nucleic acid includes these motifs. Thus in some embodiments of the invention the T rich nucleic acids include CpG dinucleotides and in other embodiments the T rich nucleic acids are free of CpG dinucleotides. The CpG dinucleotides may be methylated or unmethylated.

Examples of T rich nucleic acids that are free of CpG nucleic acids include but are not limited to those listed in Table 1, such as SEQ ID Nos: 59-63, 73-75, 142, 215, 226, 241, 267-269, 282, 301, 304, 330, 342, 358, 370-372, 393, 433, 471, 479, 486, 491, 497, 503, 556-558, 567, 694, 793-794, 797, 833, 852, 861, 867, 868, 882, 886, 905, 907, 908, and 910-913. Examples of T rich nucleic acids that include CpG nucleic acids include but are not limited to those listed in Table 1, such as SEQ ID Nos: 64, 98, 112, 146, 185, 204, 208, 214, 224, 233, 244, 246, 247, 258, 262, 263, 265, 270-273, 300, 305, 316, 317, 343, 344, 350, 352, 354, 374, 376, 392, 407, 411-413, 429-432, 434, 435, 443, 474, 475, 498-501, 518, 687, 692, 693, 804, 862, 883, 884, 888, 890, and 891.

Poly G containing nucleic acids are also useful in accordance with the invention. A “poly G nucleic acid” or “poly G antiangiogenic nucleic acid” is a nucleic acid which includes at least one poly G sequence and/or which has a nucleotide composition of greater than 25% G nucleotide residues and which inhibits angiogenesis. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 describe the properties of poly G nucleic acids.

Poly G nucleic acids preferably are nucleic acids having the following formulas:

5′ X₁X₂GGGX₃X₄ 3′

wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG 3′, or 5′ GGGNGGGNGGG 3′ wherein N represents between 0 and 20 nucleotides. In other embodiments the Poly G nucleic acid is free of unmethylated CG dinucleotides, such as, for example, the nucleic acids listed below as SEQ ID Nos: 5, 6, 73, 215, 267-269, 276, 282, 288, 297-299, 355, 359, 386, 387, 444, 476, 531, 557-559, 733, 768, 795, 796, 914-925, 928-931, 933-936, and 938. In other embodiments the poly G nucleic acid includes at least one unmethylated CG dinucleotide, such as, for example, the nucleic acids listed above as SEQ ID Nos; 67, 80-82, 141, 147, 148, 173, 178, 183, 185, 214, 224, 264, 265, 315, 329, 434, 435, 475, 519, 521-524, 526, 527, 535, 554, 565, 609, 628, 660, 661, 662, 725, 767, 825, 856, 857, 876, 892, 909, 926, 927, 932, and 937.

The antiangiogenic nucleic acids of the invention can also be those which do not possess CpG, poly-G, or T-rich motifs.

Nucleic acids having modified backbones, such as phosphorothioate backbones, also fall within the class of immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner.

The antiangiogenic nucleic acid molecules may be any size of at least 6 nucleotides but in some embodiments are in the range of between 6 and 100 or in some embodiments between 8 and 35 nucleotides in size. Nucleic acids can be produced on a large scale in plasmids. These may be administered in plasmid form or alternatively they can be degraded into oligonucleotides before administration.

“Palindromic sequence” shall mean an inverted repeat (i.e. a sequence such as ABCDEE′D′C′B′A′ in which A and A′ are bases capable of forming the usual Watson-Crick base pairs and which includes at least 6 nucleotides in the palindrome. In vivo, such sequences may form double-stranded structures. In one embodiment the nucleic acid contains a palindromic sequence. In some embodiments when the nucleic acid is a CpG nucleic acid, a palindromic sequence used in this context refers to a palindrome in which the CpG is part of the palindrome, and optionally is the center of the palindrome. In another embodiment the nucleic acid is free of a palindrome. A nucleic acid that is free of a palindrome does not have any regions of 6 nucleotides or greater in length which are palindromic. A nucleic acid that is free of a palindrome can include a region of less than 6 nucleotides which are palindromic.

A “stabilized nucleic acid molecule” shall mean a nucleic acid molecule that is relatively resistant to in vivo degradation (e.g. via an exo- or endo-nuclease). Stabilization can be a function of length or secondary structure. Nucleic acids that are tens to hundreds of kbs long are relatively resistant to in vivo degradation. For shorter nucleic acids, secondary structure can stabilize and increase their effect. For example, if the 3′ end of an oligonucleotide has self-complementarity to an upstream region, so that it can fold back and form a sort of stem loop structure, then the oligonucleotide becomes stabilized and therefore exhibits more activity.

Some stabilized oligonucleotides of the instant invention have a modified backbone. It has been demonstrated that modification of the oligonucleotide backbone provides enhanced activity of the nucleic acids when administered in vivo. Nucleic acids, including at least two phosphorothioate linkages at the 5′ end of the oligonucleotide and multiple phosphorothioate linkages at the 3′ end, preferably 5, may provide maximal activity and protect the oligonucleotide from degradation by intracellular exo- and endo-nucleases. Other modified oligonucleotides include phosphodiester modified oligonucleotide, combinations of phosphodiester and phosphorothioate oligonucleotide, methylphosphonate, methylphosphorothioate, phosphorodithioate, and combinations thereof. Each of these combinations and their particular effects on immune cells is discussed in more detail in PCT Published Patent Applications claiming priority to U.S. Pat. Nos. 6,207,646B1 and 6,239,116B1, the entire contents of which are hereby incorporated by reference. It is believed that these modified oligonucleotides may show more antiangiogenic activity due to enhanced nuclease resistance, increased cellular uptake, increased protein binding, and/or altered intracellular localization.

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

For use in vivo, nucleic acids are preferably relatively resistant to degradation (e.g., via endo-and exo-nucleases). Secondary structures, such as stem loops, can stabilize nucleic acids against degradation. Alternatively, nucleic acid stabilization can be accomplished via phosphate backbone modifications. One type of stabilized nucleic acid has at least a partial phosphorothioate modified backbone. Phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Other sources of nucleic acids useful according to the invention include standard viral and bacterial vectors, many of which are commercially available. In its broadest sense, a “vector” is any nucleic acid material which is ordinarily used to deliver and facilitate the transfer of nucleic acids to cells. The vector as used herein may be an empty vector or a vector carrying a gene which can be expressed. In the case when the vector is carrying a gene the vector generally transports the gene to the target cells with reduced degradation relative to the extent of degradation that would result in the absence of the vector. In this case the vector optionally includes gene expression sequences to enhance expression of the gene in target cells such as immune cells, but it is not required that the gene be expressed in the cell.

In general, vectors include, but are not limited to, plasmids, phagemids, viruses, other vehicles derived from viral or bacterial sources. Viral vectors are one type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Some viral vectors are based on non-cytopathic eukaryotic viruses in which non-essential genes have been replaced with a nucleic acid to be delivered. Non-cytopathic viruses include retroviruses, the life cycle of which involves reverse transcription of genomic viral RNA into DNA.

Standard protocols for producing empty vectors or vectors carrying genes (including the steps of incorporation of exogenous genetic material into a plasmid, transfection of a packaging cell line with plasmid, production of recombinant retroviruses by the packaging cell line, collection of viral particles from tissue culture media, and/or infection of the target cells with viral particles) are provided in Kriegler, M., “Gene Transfer and Expression, A Laboratory Manual,” W. H. Freeman C.O., New York (1990) and Murry, E. J. Ed. “Methods in Molecular Biology,” vol. 7, Humana Press, Inc., Cliffton, N.J. (1991).

Other vectors include plasmid vectors. Plasmid vectors have been extensively described in the art and are well-known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. Some plasmids, however, having a promoter compatible with the host cell, can express a peptide from a gene operatively encoded within the plasmid. Some commonly used plasmids include pBR322, pUC18, pUC19, pcDNA3.1, SV40, and pBlueScript. Other plasmids are well-known to those of ordinary skill in the art. Additionally, plasmids may be custom designed using restriction enzymes and ligation reactions to remove and add specific fragments of DNA.

Exemplary antiangiogenic nucleic acid sequences include but are not limited to those antiangiogenic sequences shown in Table 1 (SEQ ID NO: 1 to SEQ ID NO:1093). The Table lists the SEQ ID NO, nucleotide sequence of the oligonucleotide (ODN sequence), and backbone modification, if any.

Backbone modifications are abbreviated as follows:

S=phosphorothioate

O=phosphodiester

SOS=phosphorothioate and phosphodiester chimeric with phosphodiester in middle

SO=phosphorothioate and phosphodiester chimeric with phosphodiester on 3′ end

OS=phosphorothioate and phosphodiester chimeric with phosphodiester on 5′ end

S2=phosphorodithioate

S2O=phosphorodithioate and phosphodiester chimeric with phosphodiester on 3′ end

OS2=phosphorodithioate and phosphodiester chimeric with phosphodiester on 5′ end

X=unknown

p-ethoxy=p-ethoxy backbone; see, e.g., U.S. Pat. No. 6,015,886

PO=phosphodiester

ODN sequence symbols, other than a, c, g and t, are as follows:

i=inosine

n=a, c, g, or t

d=a,g or t

h=a, c or t

b=c, g or t; if “b” is single and is listed on 5′ or 3′ end of oligonucleotide, then “b” indicates a biotin moiety attached to that end of the oligonucleotide

q=5-methyl-cytosine

m=a or c

s=c or g

x=if “x” is single and is listed on 5′ or 3′ end of oligonucleotide, then “x” indicates a biotin moiety attached to that end of the oligonucleotide

z=5-methyl-cytidine

f=FITC moiety attached to 5′ or 3′ end of oligonucleotide

TABLE 1

| SEQ ID NO: | ODN SEQUENCE | BACKBONE |
|---|---|---|
| 1 | tctcccagcgtgcgccat | S |
| 2 | ataatccagcttgaaccaag | S |
| 3 | ataatcgacgttcaagcaag | S |
| 4 | taccgcgtgcgaccctct | S |
| 5 | ggggagggt | S |
| 6 | ggggagggg | S |
| 7 | ggtgaggtg | S |
| 8 | tccatgtzgttcctgatgct | o |
| 9 | gctaccttagzgtga | o |
| 10 | tccatgazgttcctgatgct | o |
| 11 | tccatgacgttcztgatgct | o |
| 12 | gctagazgttagtgt | o |
| 13 | agctccatggtgctcactg | s |
| 14 | ccacgtcgaccctcaggcga | s |
| 15 | gcacatcgtcccgcagccga | s |
| 16 | gtcactcgtggtacctcga | s |
| 17 | gttggatacaggccagactttgttg | o |
| 18 | gattcaacttgcgctcatcttaggc | o |
| 19 | accatggacgaactgtttcccctc | s |
| 20 | accatggacgagctgtttcccctc | s |
| 21 | accatggacgacctgtttcccctc | s |
| 22 | accatggacgtactgtttcccctc | s |
| 23 | accatggacggtctgtttcccctc | s |
| 24 | accatggacgttctgtttcccctc | s |
| 25 | ccactcacatctgctgctccacaag | o |
| 26 | acttctcatagtccctttggtccag | o |
| 27 | tccatgagcttcctgagtct | o |
| 28 | gaggaaggigiggaigacgt | o |
| 29 | gtgaaticgttcicgggict | o |
| 30 | aaaaaa | s |
| 31 | cccccc | s |
| 32 | ctgtca | s |
| 33 | tcgtag | s |
| 34 | tcgtgg | s |
| 35 | cgtcgt | s |
| 36 | tccatgtcggtcctgagtct | sos |
| 37 | tccatgccggtcctgagtct | sos |
| 38 | tccatgacggtcctgagtct | sos |
| 39 | tccatgacggtcctgagtct | sos |
| 40 | tccatgtcgatcctgagtct | sos |
| 41 | tccatgtcgctcctgagtct | sos |
| 42 | tccatgtcgttcctgagtct | sos |
| 43 | tccatgacgttcctgagtct | sos |
| 44 | tccataacgttcctgagtct | sos |
| 45 | tccatgacgtccctgagtct | sos |
| 46 | tccatcacgtgcctgagtct | sos |
| 47 | tccatgctggtcctgagtct | sos |
| 48 | tccatgtzggtcctgagtct | sos |
| 49 | ccgcttcctccagatgagctcatgggtttctccaccaag | O |
| 50 | cttggtggagaaacccatgagctcatctggaggaagcgg | O |
| 51 | ccccaaagggatgagaagtt | O |
| 52 | agatagcaaatcggctgacg | O |
| 53 | ggttcacgtgctcatggctg | O |
| 54 | tctcccagcgtgcgccat | S |
| 55 | tctcccagcgtgcgccat | S |
| 56 | taccgcgtgcgaccctct | S |
| 57 | ataatccagcttgaaccaag | S |
| 58 | ataatcgacgttcaagcaag | S |
| 59 | tccatgattttcctgatttt | O |
| 60 | ttgtttttttgtttttttgttttt | S |
| 61 | ttttttttgtttttttgttttt | O |
| 62 | tgctgcttttgtgcttttgtgctt | S |
| 63 | tgctgcttgtgcttttgtgctt | O |
| 64 | gcattcatcaggcgggcaagaat | O |
| 65 | taccgagcttcgacgagatttca | O |
| 66 | gcatgacgttgagct | s |
| 67 | cacgttgaggggcat | s |
| 68 | ctgctgagactggag | s |
| 69 | tccatgacgttcctgacgtt | s |
| 70 | gcatgagcttgagctga | O |
| 71 | tcagcgtgcgcc | S |
| 72 | atgacgttcctgacgtt | s |
| 73 | ttttggggttttggggtttt | s |
| 74 | tctaggctttttaggcttcc | S |
| 75 | tgcattttttaggccaccat | S |
| 76 | tctcccagcgtgcgtgcgccat | s |
| 77 | tctcccagcgggcgcat | s |
| 78 | tctcccagcgagcgccat | s |
| 79 | tctcccagcgcgcgccat | s |
| 80 | ggggtgacgttcagggggg | sos |
| 81 | ggggtccagcgtgcgccatggggg | sos |
| 82 | ggggtgtcgttcagggggg | sos |
| 83 | tccatgtcgttcctgtcgtt | s |
| 84 | tccatagcgttcctagcgtt | s |
| 85 | tcgtcgctgtctccgcttctt | s |
| 86 | gcatgacgttgagct | Sos |
| 87 | tctcccagcgtgcgccatat | Sos |
| 88 | tccatgazgttcctgazgtt | S |
| 89 | gcatgazgttgagct | O |
| 90 | tccagcgtgcgccata | sos |
| 91 | tctcccagcgtgcgccat | O |
| 92 | tccatgagcttcctgagtct | O |
| 93 | gcatgtcgttgagct | sos |
| 94 | tcctgacgttcctgacgtt | s |
| 95 | gcatgatgttgagct | o |
| 96 | gcatttcgaggagct | o |
| 97 | gcatgtagctgagct | o |
| 98 | tccaggacgttcctagttct | o |
| 99 | tccaggagcttcctagttct | o |
| 100 | tccaggatgttcctagttct | o |
| 101 | tccagtctaggcctagttct | o |
| 102 | tccagttcgagcctagttct | o |
| 103 | gcatggcgttgagct | sos |
| 104 | gcatagcgttgagct | sos |
| 105 | gcattgcgttgagct | sos |
| 106 | gcttgcgttgcgttt | sos |
| 107 | tctcccagcgttgcgccatat | sos |
| 108 | tctcccagcgtgcgttatat | sos |
| 109 | tctccctgcgtgcgccatat | sos |
| 110 | tctgcgtgcgtgcgccatat | sos |
| 111 | tctcctagcgtgcgccatat | sos |
| 112 | tctcccagcgtgcgcctttt | sos |
| 113 | gctandcghhagc | o |
| 114 | tcctgacgttccc | o |
| 115 | ggaagacgttaga | o |
| 116 | tcctgacgttaga | o |
| 117 | tcagaccagctggtcgggtgttcctga | o |
| 118 | tcaggaacacccgaccagctggtctga | o |
| 119 | gctagtcgatagc | o |
| 120 | gctagtcgctagc | o |
| 121 | gcttgacgtctagc | o |
| 122 | gcttgacgtttagc | o |
| 123 | gcttgacgtcaagc | o |
| 124 | gctagacgtttagc | o |
| 125 | tccatgacattcctgatgct | o |
| 126 | gctagacgtctagc | o |
| 127 | ggctatgtcgttcctagcc | o |
| 128 | ggctatgtcgatcctagcc | o |
| 129 | ctcatgggtttctccaccaag | o |
| 130 | cttggtggagaaacccatgag | o |
| 131 | tccatgacgttcctagttct | o |
| 132 | ccgcttcctccagatgagctcatg | o |
| 133 | catgagctcatctggaggaagcgg | o |
| 134 | ccagatgagctcatgggtttctcc | o |
| 135 | ggagaaacccatgagctcatctgg | o |
| 136 | agcatcaggaacgacatgga | o |
| 137 | tccatgacgttcctgacgtt | RNA |
| 138 | gcgcgcgcgcgcgcgcgcg | o |
| 139 | ccggccggccggccggccgg | o |
| 140 | ttccaatcagccccacccgctctggccccaccctcaccctcca | o |
| 141 | tggagggtgagggtggggccagagcgggtggggctgattggaa | o |
| 142 | tcaaatgtgggattttcccatgagtct | o |
| 143 | agactcatgggaaaatcccacatttga | o |
| 144 | tgccaagtgctgagtcactaataaaga | o |
| 145 | tctttattagtgactcagcacttggca | o |
| 146 | tgcaggaagtccgggttttccccaacccccc | o |
| 147 | ggggggttggggaaaacccggacttcctgca | o |
| 148 | ggggactttccgctggggactttccagggggactttcc | Sos |
| 149 | tccatgacgttcctctccatgacgttcctctccatgacgttcctc | o |
| 150 | gaggaacgtcatggagaggaacgtcatggagaggaacgtcatgga | o |
| 151 | ataatagagcttcaagcaag | s |
| 152 | tccatgacgttcctgacgtt | s |
| 153 | tccatgacgttcctgacgtt | sos |
| 154 | tccaggactttcctcaggtt | s |
| 155 | tcttgcgatgctaaaggacgtcacattgcacaatcttaataaggt | o |
| 156 | accttattaagattgtgcaatgtgacgtcctttagcatcgcaaga | o |
| 157 | tcctgacgttcctggcggtcctgtcgct | o |
| 158 | tcctgtcgctcctgtcgct | o |
| 159 | tcctgacgttgaagt | o |
| 160 | tcctgtcgttgaagt | o |
| 161 | tcctggcgttgaagt | o |
| 162 | tcctgccgttgaagt | o |
| 163 | tccttacgttgaagt | o |
| 164 | tcctaacgttgaagt | o |
| 165 | tcctcacgttgaagt | o |
| 166 | tcctgacgatgaagt | o |
| 167 | tcctgacgctgaagt | o |
| 168 | tcctgacggtgaagt | o |
| 169 | tcctgacgtagaagt | o |
| 170 | tcctgacgtcgaagt | o |
| 171 | tcctgacgtggaagt | o |
| 172 | tcctgagcttgaagt | o |
| 173 | gggggacgttggggg | o |
| 174 | tcctgacgttccttc | o |
| 175 | tctcccagcgagcgagcgccat | s |
| 176 | tcctgacgttcccctggcggtcccctgtcgct | O |
| 177 | tcctgtcgctcctgtcgctcctgtcgct | O |
| 178 | tcctggcggggaagt | o |
| 179 | tcctgazgttgaagt | o |
| 180 | tcztgacgttgaagt | o |
| 181 | tcctagcgttgaagt | o |
| 182 | tccagacgttgaagt | o |
| 183 | tcctgacggggaagt | o |
| 184 | tcctggcggtgaagt | o |
| 185 | ggctccggggagggaatttttgtctat | o |
| 186 | atagacaaaaattccctccccggagcc | o |
| 187 | tccatgagcttccttgagtct | RNA |
| 188 | tcgtcgctgtctccgcttctt | so |
| 189 | tcgtcgctgtctccgcttctt | s20 |
| 190 | tcgagacattgcacaatcatctg | O |
| 191 | cagattgtgcaatgtctcga | O |
| 192 | tccatgtcgttcctgatgcg | O |
| 193 | gcgatgtcgttcctgatgct | O |
| 194 | gcgatgtcgttcctgatgcg | O |
| 195 | tccatgtcgttccgcgcgcg | O |
| 196 | tccatgtcgttcctgccgct | O |
| 197 | tccatgtcgttcctgtagct | O |
| 198 | gcggcgggcggcgcgcgccc | O |
| 199 | atcaggaacgtcatgggaagc | O |
| 200 | tccatgagcttcctgagtct | p-ethoxy |
| 201 | tcaacgtt | p-ethoxy |
| 202 | tcaagctt | p-ethoxy |
| 203 | tcctgtcgttcctgtcgtt | S |
| 204 | tccatgtcgtttttgtcgtt | S |
| 205 | tcctgtcgttccttgtcgtt | S |
| 206 | tccttgtcgttcctgtcgtt | S |
| 207 | btccattccatgacgttcctgatgcttcca | os |
| 208 | tcctgtcgttttttgtcgtt | S |
| 209 | tcgtcgctgtctccgcttctt | S |
| 210 | tcgtcgctgtctgcccttctt | s |
| 211 | tcgtcgctgttgtcgtttctt | s |
| 212 | tcctgtcgttcctgtcgttggaacgacagg | o |
| 213 | tcctgtcgttcctgtcgtttcaacgtcaggaacgacagga | o |
| 214 | ggggtctgtcgttttgggggg | sos |
| 215 | ggggtctgtgcttttgggggg | sos |
| 216 | tccggccgttgaagt | o |
| 217 | tccggacggtgaagt | o |
| 218 | tcccgccgttgaagt | o |
| 219 | tccagacggtgaagt | o |
| 220 | tcccgacggtgaagt | o |
| 221 | tccagagcttgaagt | o |
| 222 | tccatgtzgttcctgtzgtt | s |
| 223 | tccatgacgttcctgacgtt | sos |
| 224 | ggggttgacgttttgggggg | sos |
| 225 | tccaggacttctctcaggtt | s |
| 226 | tttttttttttttttttttt | s |
| 227 | tccatgccgttcctgccgtt | s |
| 228 | tccatggcgggcctggcggg | s |
| 229 | tccatgacgttcctgccgtt | s |
| 230 | tccatgacgttcctggcggg | s |
| 231 | tccatgacgttcctgcgttt | s |
| 232 | tccatgacggtcctgacggt | s |
| 233 | tccatgcgtgcgtgcgtttt | s |
| 234 | tccatgcgttgcgttgcgtt | s |
| 235 | Btccattccattctaggcctgagtcttccat | os |
| 236 | tccatagcgttcctagcgtt | o |
| 237 | tccatgtcgttcctgtcgtt | o |
| 238 | tccatagcgatcctagcgat | o |
| 239 | tccattgcgttccttgcgtt | o |
| 240 | tccatagcggtcctagcggt | o |
| 241 | tccatgattttcctgcagttcctgatttt | |
| 242 | tccatgacgttcctgcagttcctgacgtt | s |
| 243 | ggcggcggcggcggcggcgg | O |
| 244 | tccacgacgttttcgacgtt | S |
| 245 | tcgtcgttgtcgttgtcgtt | S |
| 246 | tcgtcgttttgtcgttttgtcgtt | S |
| 247 | tcgtcgttgtcgttttgtcgtt | S |
| 248 | gcgtgcgttgtcgttgtcgtt | S |
| 249 | czggczggczgggczccgg | o |
| 250 | gcggcgggcggcgcgcgccc | S |
| 251 | agicccgigaacgiattcac | o |
| 252 | tgtcgtttgtcgtttgtcgtt | S |
| 253 | tgtcgttgtcgttgtcgttgtcgtt | S |
| 254 | tgtcgttgtcgttgtcgttgtcgtt | S |
| 255 | tcgtcgtcgtcgtt | s |
| 256 | tgtcgttgtcgtt | s |
| 257 | cccccccccccccccccccc | s |
| 258 | tctagcgtttttagcgttcc | sos |
| 259 | tgcatcccccaggccaccat | s |
| 26O | tcgtcgtcgtcgtcgtcgtcgtt | sos |
| 261 | tcgtcgttgtcgttgtcgtt | sos |
| 262 | tcgtcgttttgtcgttttgtcgtt | sos |
| 263 | tcgtcgttgtcgttttgtcgtt | sos |
| 264 | ggggagggaggaacttcttaaaattcccccagaatgttt | o |
| 265 | aaacattctgggggaattttaagaagttcctccctcccc | o |
| 266 | atgtttacttcttaaaattcccccagaatgttt | o |
| 267 | aaacattctgggggaattttaagaagtaaacat | o |
| 268 | atgtttactagacaaaattcccccagaatgttt | o |
| 269 | aaacattctgggggaattttgtctagtaaacat | o |
| 270 | aaaattgacgttttaaaaaa | sos |
| 271 | ccccttgacgttttcccccc | sos |
| 272 | ttttcgttgtttttgtcgtt | |
| 273 | tcgtcgttttgtcgttttgtcgtt | sos |
| 274 | ctgcagcctgggac | o |
| 275 | acccgtcgtaattatagtaaaaccc | o |
| 276 | ggtacctgtggggacattgtg | o |
| 277 | agcaccgaacgtgagagg | o |
| 278 | tccatgccgttcctgccgtt | o |
| 279 | tccatgacggtcctgacggt | o |
| 280 | tccatgccggtcctgccggt | o |
| 281 | tccatgcgcgtcctgcgcgt | o |
| 282 | ctggtctttctggtttttttctgg | s |
| 283 | tcaggggtggggggaacctt | sos |
| 284 | tccatgazgttcctagttct | o |
| 285 | tccatgatgttcctagttct | o |
| 286 | cccgaagtcatttcctcttaacctgg | o |
| 287 | ccaggttaagaggaaatgacttcggg | o |
| 288 | tcctggzggggaagt | o |
| 289 | gzggzgggzggzgzgzgccc | x |
| 290 | tccatgtgcttcctgatgct | o |
| 291 | tccatgtccttcctgatgct | |
| 292 | tccatgtcgttcctagttct | |
| 293 | tccaagtagttcctagttct | o |
| 294 | tccatgtagttcctagttct | o |
| 295 | tcccgcgcgttccgcgcgtt | s |
| 296 | tcctggcggtcctggcggtt | s |
| 297 | tcctggaggggaagt | o |
| 298 | tcctgggggggaagt | o |
| 299 | tcctggtggggaagt | o |
| 300 | tcgtcgttttgtcgttttgtcgtt | o |
| 301 | ctggtctttctggtttttttctgg | o |
| 302 | tccatgacgttcctgacgtt | o |
| 303 | tccaggacttctctcaggtt | sos |
| 304 | tzgtzgttttgtzgttttgtzgtt | o |
| 305 | btcgtcgttttgtcgttttgtcgttttttt | os |
| 306 | gctatgacgttccaaggg | s |
| 307 | tcaacgtt | s |
| 308 | tccaggactttcctcaggtt | o |
| 309 | ctctctgtaggcccgcttgg | s |
| 310 | ctttccgttggacccctggg | s |
| 311 | gtccgggccaggccaaagtc | s |
| 312 | gtgcgcgcgagcccgaaatc | s |
| 313 | tccatgaigttcctgaigtt | s |
| 314 | aatagtcgccataacaaaac | o |
| 315 | aatagtcgccatggcggggc | o |
| 316 | btttttccatgtcgttcctgatgcttttt | os |
| 317 | tcctgtcgttgaagtttttt | o |
| 318 | gctagctttagagctttagagctt | o |
| 319 | tgctgcttcccccccccccc | o |
| 320 | tcgacgttcccccccccccc | o |
| 321 | tcgtcgttcccccccccccc | o |
| 322 | tcgtcgttcccccccccccc | o |
| 323 | tcgccgttcccccccccccc | o |
| 324 | tcgtcgatcccccccccccc | o |
| 325 | tcctgacgttgaagt | s |
| 326 | tcctgccgttgaagt | s |
| 327 | tcctgacggtgaagt | s |
| 328 | tcctgagcttgaagt | s |
| 329 | tcctggcggggaagt | s |
| 330 | aaaatctgtgcttttaaaaaa | sos |
| 331 | gatccagtcacagtgacctggcagaatctggat | o |
| 332 | gatccagattctgccaggtcactgtgactggat | o |
| 333 | gatccagtcacagtgactcagcagaatctggat | o |
| 334 | gatccagattctgctgagtcactgtgactggat | o |
| 335 | tcgtcgttccccccczcccc | o |
| 336 | tzgtqgttcccccccccccc | o |
| 337 | tzgtcgttcccccccccccc | o |
| 338 | tcgtzgttcccccccccccc | o |
| 339 | tcgtcgctcccccccccccc | o |
| 340 | tcgtcggtcccccccccccc | o |
| 341 | tcggcgttcccccccccccc | o |
| 342 | ggccttttcccccccccccc | o |
| 343 | tcgtcgttttgacgttttgtcgtt | s |
| 344 | tcgtcgttttgacgttttgacgtt | s |
| 345 | ccgtcgttcccccccccccc | o |
| 346 | gcgtcgttcccccccccccc | o |
| 347 | tcgtcattcccccccccccc | o |
| 348 | acgtcgttcccccccccccc | o |
| 349 | ctgtcgttcccccccccccc | o |
| 350 | btttttcgtcgttcccccccccccc | os |
| 351 | tcgtcgttccccccccccccb | o |
| 352 | tcgtcgttttgtcgttttgtcgttb | o |
| 353 | tccagttccttcctcagtct | o |
| 354 | tzgtcgttttgtcgttttgtcgtt | o |
| 355 | tcctggaggggaagt | s |
| 356 | tcctgaaaaggaagt | s |
| 357 | tcgtcgttccccccccc | s |
| 358 | tzgtzgttttgtzgttttgtzgtt | s |
| 359 | ggggtcaagcttgagggggg | sos |
| 360 | tgctgcttcccccccccccc | s |
| 361 | tcgtcgtcgtcgtt | s2 |
| 362 | tcgtcgtcgtcgtt | s20 |
| 363 | tcgtcgtcgtcgtt | os2 |
| 364 | tcaacgttga | s |
| 365 | tcaacgtt | s |
| 366 | atagttttccatttttttac | |
| 367 | aatagtcgccatcgcgcgac | o |
| 368 | aatagtcgccatcccgggac | o |
| 369 | aatagtcgccatcccccccc | o |
| 370 | tgctgcttttgtgcttttgtgctt | o |
| 371 | ctgtgctttctgtgtttttctgtg | s |
| 372 | ctaatctttctaatttttttctaa | s |
| 373 | tcgtcgttggtgtcgttggtgtcgtt | s |
| 374 | tcgtcgttggttgtcgttttggtt | s |
| 375 | accatggacgagctgtttcccctc | |
| 376 | tcgtcgttttgcgtgcgttt | s |
| 377 | ctgtaagtgagcttggagag | |
| 378 | gagaacgctggaccttcc | |
| 379 | cgggcgactcagtctatcgg | |
| 380 | gttctcagataaagcggaaccagcaacagacacagaa | |
| 381 | ttctgtgtctgttgctggttccgctttatctgagaac | |
| 382 | cagacacagaagcccgatagacg | |
| 383 | agacagacacgaaacgaccg | |
| 384 | gtctgtcccatgatctcgaa | |
| 385 | gctggccagcttacctcccg | |
| 386 | ggggcctctatacaacctggg | |
| 387 | ggggtccctgagactgcc | |
| 388 | gagaacgctggaccttccat | |
| 389 | tccatgtcggtcctgatgct | |
| 390 | ctcttgcgacctggaaggta | |
| 391 | aggtacagccaggactacga | |
| 392 | accatggacgacctgtttcccctc | |
| 393 | accatggattacctttttcccctt | |
| 394 | atggaaggtccagcgttctc | o |
| 395 | agcatcaggaccgacatgga | o |
| 396 | ctctccaagctcacttacag | |
| 397 | tccctgagactgccccacctt | |
| 398 | gccaccaaaacttgtccatg | |
| 399 | gtccatggcgtgcgggatga | |
| 400 | cctctatacaacctgggac | |
| 401 | cgggcgactcagtctatcgg | |
| 402 | gcgctaccggtagcctgagt | |
| 403 | cgactgccgaacaggatatcggtgatcagcactgg | |
| 404 | ccagtgctgatcaccgatatcctgttcggcagtcg | |
| 405 | ccaggttgtatagaggc | |
| 406 | tctcccagcgtacgccat | s |
| 407 | tctcccagcgtgcgtttt | s |
| 408 | tctcccgacgtgcgccat | s |
| 409 | tctcccgtcgtgcgccat | s |
| 410 | ataatcgtcgttcaagcaag | s |
| 411 | tcgtcgttttgtcgttttgtcgt | s2 |
| 412 | tcgtcgttttgtcgttttgtcgtt | s2 |
| 413 | tcgtcgttttgtcgttttgtcgtt | s2 |
| 414 | tcntcgtnttntcgtnttntcgtn | s |
| 415 | tctcccagcgtcgccat | s |
| 416 | tctcccatcgtcgccat | s |
| 417 | ataatcgtgcgttcaagaaag | s |
| 418 | ataatcgacgttcccccccc | s |
| 419 | tctatcgacgttcaagcaag | s |
| 420 | tcc tga cgg gg agt | s |
| 421 | tccatgacgttcctgatcc | |
| 422 | tccatgacgttcctgatcc | |
| 423 | tccatgacgttcctgatcc | |
| 424 | tcc tgg cgt gga agt | s |
| 425 | tccatgacgttcctgatcc | |
| 426 | tcgtcgctgttgtcgtttctt | s |
| 427 | agcagctttagagctttagagctt | s |
| 428 | cccccccccccccccccccccccc | s |
| 429 | tcgtcgttttgtcgttttgtcgttttgtcgtt | s |
| 430 | tcgtcgttttttgtcgttttttgtcgtt | s |
| 431 | tcgtcgtttttttttttttt | s |
| 432 | tttttcaacgttgatttttt | sos |
| 433 | tttttttttttttttttttttttt | S |
| 434 | ggggtcgtcgttttgggggg | |
| 435 | tcgtcgttttgtcgttttgggggg | |
| 436 | tcgtcgctgtctccgcttcttcttgcc | s |
| 437 | tcgtcgctgtctccg | s |
| 438 | ctgtaagtgagcttggagag | |
| 439 | gagaacgctggaccttccat | |
| 440 | ccaggttgtatagaggc | |
| 441 | gctagacgttagcgtga | |
| 442 | ggagctcttcgaacgccata | |
| 443 | tctccatgatggttttatcg | |
| 444 | aaggtggggcagtctcaggga | |
| 445 | atcggaggactggcgcgccg | |
| 446 | ttaggacaaggtctagggtg | |
| 447 | accacaacgagaggaacgca | |
| 448 | ggcagtgcaggctcaccggg | |
| 449 | gaaccttccatgctgtt | |
| 450 | gctagacgttagcgtga | |
| 451 | gcttggagggcctgtaagtg | |
| 452 | gtagccttccta | |
| 453 | cggtagccttccta | |
| 454 | cacggtagccttccta | |
| 455 | agcacggtagccttccta | |
| 456 | gaacgctggaccttccat | |
| 457 | gaccttccat | |
| 458 | tggaccttccat | |
| 459 | gctggaccttccat | |
| 460 | acgctggaccttccat | |
| 461 | taagctctgtcaacgccagg | |
| 462 | gagaacgctggaccttccatgt | |
| 463 | tccatgtcggtcctgatgct | |
| 464 | ttcatgccttgcaaaatggcg | |
| 465 | tgctagctgtgcctgtacct | |
| 466 | agcatcaggaccgacatgga | |
| 467 | gaccttccatgtcggtcctgat | |
| 468 | acaaccacgagaacgggaac | |
| 469 | gaaccttccatgctgttccg | |
| 470 | caatcaatctgaggagaccc | |
| 471 | tcagctctggtactttttca | |
| 472 | tggttacggtctgtcccatg | |
| 473 | gtctatcggaggactggcgc | |
| 474 | cattttacgggcgggcgggc | |
| 475 | gaggggaccattttacgggc | |
| 476 | tgtccagccgaggggaccat | |
| 477 | cgggcttacggcggatgctg | |
| 478 | tggaccttctatgtcggtcc | |
| 479 | tgtcccatgtttttagaagc | |
| 480 | gtggttacggtcgtgcccat | |
| 481 | cctccaaatgaaagaccccc | |
| 482 | ttgtactctccatgatggtt | |
| 483 | ttccatgctgttccggctgg | |
| 484 | gaccttctatgtcggtcctg | |
| 485 | gagaccgctcgaccttcgat | |
| 486 | ttgccccatattttagaaac | |
| 487 | ttgaaactgaggtgggac | |
| 488 | ctatcggaggactggcgcgcc | |
| 489 | cttggagggcctcccggcgg | |
| 490 | gctgaaccttccatgctgtt | |
| 491 | tagaaacagcattcttcttttagggcagcaca | |
| 492 | agatggttctcagataaagcggaa | |
| 493 | ttccgctttatctgagaaccatct | |
| 494 | gtcccaggttgtatagaggctgc | |
| 495 | gcgccagtcctccgatagac | |
| 496 | atcggaggactggcgcgccg | |
| 497 | ggtctgtcccatatttttag | |
| 498 | tttttcaacgttgagggggg | sos |
| 499 | tttttcaagcgttgatttttt | sos |
| 500 | ggggtcaacgttgatttttt | sos |
| 501 | ggggttttcaacgttttgagggggg | sos |
| 502 | ggttacggtctgtcccatat | |
| 503 | ctgtcccatatttttagaca | |
| 504 | accatcctgaggccattcgg | |
| 505 | cgtctatcgggcttctgtgtctg | |
| 506 | ggccatcccacattgaaagtt | |
| 507 | ccaaatatcggtggtcaagcac | |
| 508 | gtgcttgaccaccgatatttgg | |
| 509 | gtgctgatcaccgatatcctgttcgg | |
| 510 | ggccaactttcaatgtgggatggcctc | |
| 511 | ttccgccgaatggcctcaggatggtac | |
| 512 | tatagtccctgagactgccccaccttctcaacaacc | |
| 513 | gcagcctctatacaacctgggacggga | |
| 514 | ctatcggaggactggcgcgccg | |
| 515 | tatcggaggactggcgcgccg | |
| 516 | gatcggaggactggcgcgccg | |
| 517 | ccgaacaggatatcggtgatcagcac | |
| 518 | ttttggggtcaacgttgagggggg | |
| 519 | ggggtcaacgttgagggggg | sos |
| 520 | cgcgcgcgcgcgcgcgcgcg | s |
| 521 | ggggcatgacgttcgggggg | ss |
| 522 | ggggcatgacgttcaaaaaa | s |
| 523 | ggggcatgagcttcgggggg | s |
| 524 | ggggcatgacgttcgggggg | sos |
| 525 | aaaacatgacgttcaaaaaa | sos |
| 526 | aaaacatgacgttcgggggg | sos |
| 527 | ggggcatgacgttcaaaaaa | sos |
| 528 | accatggacgatctgtttcccctc | s |
| 529 | gccatggacgaactgttccccctc | s |
| 530 | cccccccccccccccccccc | sos |
| 531 | gggggggggggggggggggg | sos |
| 532 | gctgtaaaatgaatcggccg | sos |
| 533 | ttcgggcggactcctccatt | sos |
| 534 | tatgccgcgcccggacttat | sos |
| 535 | ggggtaatcgatcagggggg | sos |
| 536 | tttgagaacgctggaccttc | sos |
| 537 | gatcgctgatctaatgctcg | sos |
| 538 | gtcggtcctgatgctgttcc | sos |
| 539 | tcgtcgtcagttcgctgtcg | sos |
| 540 | ctggaccttccatgtcgg | sos |
| 541 | gctcgttcagcgcgtct | sos |
| 542 | ctggaccttccatgtc | sos |
| 543 | cactgtccttcgtcga | sos |
| 544 | cgctggaccttccatgtcgg | sos |
| 545 | gctgagctcatgccgtctgc | sos |
| 546 | aacgctggaccttccatgtc | sos |
| 547 | tgcatgccgtacacagctct | sos |
| 548 | ccttccatgtcggtcctgat | sos |
| 549 | tactcttcggatcccttgcg | sos |
| 550 | ttccatgtcggtcctgat | sos |
| 551 | ctgattgctctctcgtga | sos |
| 552 | ggcgttattcctgactcgcc | o |
| 553 | cctacgttgtatgcgcccagct | o |
| 554 | ggggtaatcgatgagggggg | o |
| 555 | ttcgggcggactcctccatt | o |
| 556 | tttttttttttttttttttt | o |
| 557 | gggggttttttttttggggg | o |
| 558 | tttttggggggggggttttt | o |
| 559 | ggggggggggggggggggt | o |
| 560 | aaaaaaaaaaaaaaaaaaaa | o |
| 561 | cccccaaaaaaaaaaccccc | o |
| 562 | aaaaaccccccccccaaaaa | o |
| 563 | tttgaattcaggactggtgaggttgag | o |
| 564 | tttgaatcctcagcggtctccagtggc | o |
| 565 | aattctctatcggggcttctgtgtctgttgctggttccgctttat | o |
| 566 | ctagataaagcggaaccagcaacagacacagaagccccgatagag | o |
| 567 | ttttctagagaggtgcacaatgctctgg | o |
| 568 | tttgaattccgtgtacagaagcgagaagc | o |
| 569 | tttgcggccgctagacttaacctgagagata | o |
| 570 | tttgggcccacgagagacagagacacttc | o |
| 571 | tttgggcccgcttctcgcttctgtacacg | o |
| 572 | gagaacgctggaccttccat | s |
| 573 | tccatgtcggtcctgatgct | s |
| 574 | ctgtcg | s |
| 575 | tcgtga | s |
| 576 | cgtcga | s |
| 577 | agtgct | s |
| 578 | ctgtcg | o |
| 579 | agtgct | o |
| 580 | cgtcga | o |
| 581 | tcgtga | o |
| 582 | gagaacgctccagcttcgat | o |
| 583 | gctagacgtaagcgtga | o |
| 584 | gagaacgctcgaccttccat | o |
| 585 | gagaacgctggacctatccat | o |
| 586 | gctagaggttagcgtga | o |
| 587 | gagaacgctggacttccat | o |
| 588 | tcacgctaacgtctagc | o |
| 589 | bgctagacgttagcgtga | o |
| 590 | atggaaggtcgagcgttctc | o |
| 591 | gagaacgctggaccttcgat | o |
| 592 | gagaacgatggaccttccat | o |
| 593 | gagaacgctggatccat | o |
| 594 | gagaacgctccagcactgat | o |
| 595 | tccatgtcggtcctgctgat | o |
| 596 | atgtcctcggtcctgatgct | o |
| 597 | gagaacgctccaccttccat | o |
| 598 | gagaacgctggaccttcgta | o |
| 599 | batggaaggtccagcgttctc | o |
| 600 | tcctga | o |
| 601 | tcaacgtt | o |
| 602 | aacgtt | o |
| 603 | aacgttga | o |
| 604 | tcacgctaacctctagc | o |
| 605 | gagaacgctggaccttgcat | o |
| 606 | gctggaccttccat | o |
| 607 | gagaacgctggacctcatccat | o |
| 608 | gagaacgctggacgctcatccat | o |
| 609 | aacgttgaggggcat | o |
| 610 | atgcccctcaacgtt | o |
| 611 | tcaacgttga | o |
| 612 | gctggaccttccat | o |
| 613 | caacgtt | o |
| 614 | acaacgttga | o |
| 615 | tcacgt | o |
| 616 | tcaagctt | o |
| 617 | tcgtca | o |
| 618 | aggatatc | o |
| 619 | tagacgtc | o |
| 620 | gacgtcat | o |
| 621 | ccatcgat | o |
| 622 | atcgatgt | o |
| 623 | atgcatgt | o |
| 624 | ccatgcat | o |
| 625 | agcgctga | o |
| 626 | tcagcgct | o |
| 627 | ccttcgat | o |
| 628 | gtgccggggtctccgggc | s |
| 629 | gctgtggggcggctcctg | s |
| 630 | btcaacgtt | o |
| 631 | ftcaacgtt | o |
| 632 | faacgttga | o |
| 633 | tcaacgt | s |
| 634 | aacgttg | s |
| 635 | cgacga | o |
| 636 | tcaacgtt | o |
| 637 | tcgga | o |
| 638 | agaacgtt | o |
| 639 | tcatcgat | o |
| 640 | taaacgtt | s |
| 641 | ccaacgtt | s |
| 642 | gctcga | s |
| 643 | cgacgt | s |
| 644 | cgtcgt | s |
| 645 | acgtgt | s |
| 646 | cgttcg | s |
| 647 | gagcaagctggaccttccat | s |
| 648 | cgcgta | s |
| 649 | cgtacg | s |
| 650 | tcaccggt | s |
| 651 | caagagatgctaacaatgca | s |
| 652 | acccatcaatagctctgtgc | s |
| 653 | ccatcgat | o |
| 654 | tcgacgtc | o |
| 655 | ctagcgct | o |
| 656 | taagcgct | o |
| 657 | tcgcgaattcgcg | o |
| 658 | atggaaggtccagcgttct | o |
| 659 | actggacgttagcgtga | o |
| 660 | cgcctggggctggtctgg | o |
| 661 | gtgtcggggtctccgggc | o |
| 662 | gtgccggggtctccgggc | o |
| 663 | cgccgtcgcggcggttgg | o |
| 664 | gaagttcacgttgaggggcat | o |
| 665 | atctggtgagggcaagctatg | s |
| 666 | gttgaaacccgagaacatcat | s |
| 667 | gcaacgtt | o |
| 668 | gtaacgtt | o |
| 669 | cgaacgtt | o |
| 670 | gaaacgtt | o |
| 671 | caaacgtt | o |
| 672 | ctaacgtt | o |
| 673 | ggaacgtt | o |
| 674 | tgaacgtt | o |
| 675 | acaacgtt | o |
| 676 | ttaacgtt | o |
| 677 | aaaacgtt | o |
| 678 | ataacgtt | o |
| 679 | aacgttct | o |
| 680 | tccgatcg | o |
| 681 | tccgtacg | o |
| 682 | gctagacgctagcgtga | o |
| 683 | gagaacgctggacctcatcatccat | o |
| 684 | gagaacgctagaccttctat | o |
| 685 | actagacgttagtgtga | o |
| 686 | cacaccttggtcaatgtcacgt | o |
| 687 | tctccatcctatggttttatcg | o |
| 688 | cgctggaccttccat | o |
| 689 | caccaccttggtcaatgtcacgt | o |
| 690 | gctagacgttagctgga | o |
| 691 | agtgcgattgcagatcg | o |
| 692 | ttttcgttttgtggttttgtggtt | |
| 693 | ttttcgtttgtcgttttgtcgtt | |
| 694 | tttttgttttgtggttttgtggtt | |
| 695 | accgcatggattctaggcca | s |
| 696 | gctagacgttagcgt | o |
| 697 | aacgctggaccttccat | o |
| 698 | tcaazgtt | o |
| 699 | ccttcgat | o |
| 700 | actagacgttagtgtga | s |
| 701 | gctagaggttagcgtga | s |
| 702 | atggactctccagcgttctc | o |
| 703 | atcgactctcgagcgttctc | o |
| 704 | gctagacgttagc | o |
| 705 | gctagacgt | o |
| 706 | agtgcgattcgagatcg | o |
| 707 | tcagzgct | o |
| 708 | ctgattgctctctcgtga | o |
| 709 | tzaacgtt | o |
| 710 | gagaazgctggaccttccat | o |
| 711 | gctagacgttaggctga | o |
| 712 | gctacttagcgtga | o |
| 713 | gctaccttagcgtga | o |
| 714 | atcgacttcgagcgttctc | o |
| 715 | atgcactctgcagcgttctc | o |
| 716 | agtgactctccagcgttctc | o |
| 717 | gccagatgttagctgga | o |
| 718 | atcgactcgagcgttctc | o |
| 719 | atcgatcgagcgttctc | o |
| 720 | bgagaacgctcgaccttcgat | o |
| 721 | gctagacgttagctgga | sos |
| 722 | atcgactctcgagcgttctc | sos |
| 723 | tagacgttagcgtga | o |
| 724 | cgactctcgagcgttctc | o |
| 725 | ggggtcgaccttggagggggg | sos |
| 726 | gctaacgttagcgtga | o |
| 727 | cgtcgtcgt | o |
| 728 | gagaacgctggaczttccat | o |
| 729 | atcgacctacgtgcgttztc | o |
| 730 | atzgacctacgtgcgttctc | o |
| 731 | gctagazgttagcgt | o |
| 732 | atcgactctcgagzgttctc | o |
| 733 | ggggtaatgcatcagggggg | sos |
| 734 | ggctgtattcctgactgccc | s |
| 735 | ccatgctaacctctagc | o |
| 736 | gctagatgttagcgtga | o |
| 737 | cgtaccttacggtga | o |
| 738 | tccatgctggtcctgatgct | o |
| 739 | atcgactctctcgagcgttctc | o |
| 740 | gctagagcttagcgtga | o |
| 741 | atcgactctcgagtgttctc | o |
| 742 | aacgctcgaccttcgat | o |
| 743 | ctcaacgctggaccttccat | o |
| 744 | atcgacctacgtgcgttctc | o |
| 745 | gagaatgctggaccttccat | o |
| 746 | tcacgctaacctctgac | o |
| 747 | bgagaacgctccagcactgat | o |
| 748 | bgagcaagctggaccttccat | o |
| 749 | cgctagaggttagcgtga | o |
| 750 | gctagatgttaacgt | o |
| 751 | atggaaggtccacgttctc | o |
| 752 | gctagatgttagcgt | o |
| 753 | gctagacgttagtgt | o |
| 754 | tccatgacggtcctgatgct | o |
| 755 | tccatggcggtcctgatgct | o |
| 756 | gctagacgatagcgt | o |
| 757 | gctagtcgatagcgt | o |
| 758 | tccatgacgttcctgatgct | o |
| 759 | tccatgtcgttcctgatgct | o |
| 760 | gctagacgttagzgt | o |
| 761 | gctaggcgttagcgt | o |
| 762 | tccatgtzggtcctgatgct | o |
| 763 | tccatgtcggtzctgatgct | o |
| 764 | atzgactctzgagzgttctc | o |
| 765 | atggaaggtccagtgttctc | o |
| 766 | gcatgacgttgagct | o |
| 767 | ggggtcaacgttgagggggg | s |
| 768 | ggggtcaagtctgagggggg | sos |
| 769 | cgcgcgcgcgcgcgcgcgcg | o |
| 770 | cccccccccccccccccccccccccccc | s |
| 771 | ccccccccccccccccccccccccccccccccccc | s |
| 772 | tccatgtcgctcctgatcct | o |
| 773 | gctaaacgttagcgt | o |
| 774 | tccatgtcgatcctgatgct | o |
| 775 | tccatgccggtcctgatgct | o |
| 776 | aaaatcaacgttgaaaaaaa | sos |
| 777 | tccataacgttcctgatgct | o |
| 778 | tggaggtcccaccgagatcggag | o |
| 779 | cgtcgtcgtcgtcgtcgtcgt | s |
| 780 | ctgctgctgctgctgctgctg | s |
| 781 | gagaacgctccgaccttcgat | s |
| 782 | gctagatgttagcgt | s |
| 783 | gcatgacgttgagct | s |
| 784 | tcaatgctgaf | o |
| 785 | tcaacgttgaf | o |
| 786 | tcaacgttgab | o |
| 787 | gcaatattgcb | o |
| 788 | gcaatattgcf | o |
| 789 | agttgcaact | o |
| 790 | tcttcgaa | o |
| 791 | tcaacgtc | o |
| 792 | ccatgtcggtcctgatgct | o |
| 793 | gtttttatataatttggg | o |
| 794 | tttttgtttgtcgttttgtcgtt | o |
| 795 | ttggggggggtt | s |
| 796 | ggggttgggggtt | s |
| 797 | ggtggtgtaggttttgg | o |
| 798 | bgagaazgctcgaccttcgat | o |
| 799 | tcaacgttaacgttaacgtt | o |
| 800 | bgagcaagztggaccttccat | o |
| 801 | bgagaazgctccagcactgat | o |
| 802 | tcaazgttgax | o |
| 803 | gzaatattgcx | o |
| 804 | tgctgcttttgtcgttttgtgctt | o |
| 805 | ctgcgttagcaatttaactgtg | o |
| 806 | tccatgacgttcctgatgct | s |
| 807 | tgcatgccgtgcatccgtacacagctct | s |
| 808 | tgcatgccgtacacagctct | s |
| 809 | tgcatcagctct | s |
| 810 | tgcgctct | s |
| 811 | cccccccccccccccccccc | s |
| 812 | cccccccccccc | s |
| 813 | cccccccc | s |
| 814 | tgcatcagctct | sos |
| 815 | tgcatgccgtacacagctct | o |
| 816 | gagcaagctggaccttccat | s |
| 817 | tcaacgttaacgttaacgttaacgttaacgtt | s |
| 818 | gagaacgctcgaccttcgat | s |
| 819 | gtccccatttcccagaggaggaaat | o |
| 820 | ctagcggctgacgtcatcaagctag | o |
| 821 | ctagcttgatgacgtcagccgctag | o |
| 822 | cggctgacgtcatcaa | s |
| 823 | ctgacgtg | o |
| 824 | ctgacgtcat | o |
| 825 | attcgatcggggcggggcgag | o |
| 826 | ctcgccccgccccgatcgaat | o |
| 827 | gactgacgtcagcgt | o |
| 828 | ctagcggctgacgtcataaagctagc | s |
| 829 | ctagctttatgacgtcagccgctagc | s |
| 830 | ctagcggctgagctcataaagctagc | s |
| 831 | ctagtggctgacgtcatcaagctag | s |
| 832 | tccaccacgtggtctatgct | s |
| 833 | gggaatgaaagattttattataag | o |
| 834 | tctaaaaaccatctattcttaaccct | o |
| 835 | agctcaacgtcatgc | o |
| 836 | ttaacggtggtagcggtattggtc | o |
| 837 | ttaagaccaataccgctaccaccg | o |
| 838 | gatctagtgatgagtcagccggatc | o |
| 839 | gatccggctgactcatcactagatc | o |
| 840 | tccaagacgttcctgatgct | o |
| 841 | tccatgacgtccctgatgct | o |
| 842 | tccaccacgtggctgatgct | o |
| 843 | ccacgtggacctctagc | o |
| 844 | tcagaccacgtggtcgggtgttcctga | o |
| 845 | tcaggaacacccgaccacgtggtctga | o |
| 846 | catttccacgatttccca | o |
| 847 | ttcctctctgcaagagact | o |
| 848 | tgtatctctctgaaggact | o |
| 849 | ataaagcgaaactagcagcagtttc | o |
| 850 | gaaactgctgctagtttcgctttat | o |
| 851 | tgcccaaagaggaaaatttgtttcatacag | o |
| 852 | ctgtatgaaacaaattttcctctttgggca | o |
| 853 | ttagggttagggttagggtt | s |
| 854 | tccatgagcttcctgatgct | s |
| 855 | aaaacatgacgttcaaaaaa | s |
| 856 | aaaacatgacgttcgggggg | s |
| 857 | ggggcatgagcttcgggggg | sos |
| 858 | ctaggctgacgtcatcaagctagt | o |
| 859 | tctgacgtcatctgacgttggctgacgtct | o |
| 860 | ggaattagtaatagatatagaagtt | o |
| 861 | tttaccttttataaacataactaaaacaaa | o |
| 862 | gcgtttttttttgcg | s |
| 863 | atatctaatcaaaacattaacaaa | o |
| 864 | tctatcccaggtggttcctgttag | o |
| 865 | btccatgacgttcctgatgct | o |
| 866 | btccatgagcttcctgatgct | o |
| 867 | tttttttttttttf | o |
| 868 | tttttttttttttf | so |
| 869 | ctagcttgatgagctcagccgctag | o |
| 870 | ttcagttgtcttgctgcttagctaa | o |
| 871 | tccatgagcttcctgagtct | s |
| 872 | ctagcggctgacgtcatcaatctag | o |
| 873 | tgctagctgtgcctgtacct | s |
| 874 | atgctaaaggacgtcacattgca | o |
| 875 | tgcaatgtgacgtcctttagcat | o |
| 876 | gtaggggactttccgagctcgagatcctatg | o |
| 877 | cataggatctcgagctcggaaagtcccctac | o |
| 878 | ctgtcaggaactgcaggtaagg | o |
| 879 | cataacataggaatatttactcctcgc | o |
| 880 | ctccagctccaagaaaggacg | o |
| 881 | gaagtttctggtaagtcttcg | o |
| 882 | tgctgcttttgtgcttttgtgctt | s |
| 883 | tcgtcgttttgtggttttgtggtt | s |
| 884 | tcgtcgtttgtcgttttgtcgtt | s |
| 885 | tcctgacgttcggcgcgcgccc | s |
| 886 | tgctgcttttgtgcttttgtgctt | |
| 887 | tccatgagcttcctgagctt | s |
| 888 | tcgtcgtttcgtcgttttgacgtt | s |
| 889 | tcgtcgtttgcgtgcgtttcgtcgtt | s |
| 890 | tcgcgtgcgttttgtcgttttgacgtt | s |
| 891 | ttcgtcgttttgtcgttttgtcgtt | s |
| 892 | tcctgacggggaagt | s |
| 893 | tcctggcgtggaagt | s |
| 894 | tcctggcggtgaagt | s |
| 895 | tcctggcgttgaagt | s |
| 896 | tcctgacgtggaagt | s |
| 897 | gcgacgttcggcgcgcgccc | s |
| 898 | gcgacgggcggcgcgcgccc | s |
| 899 | gcggcgtgcggcgcgcgccc | s |
| 900 | gcggcggtcggcgcgcgccc | s |
| 901 | gcgacggtcggcgcgcgccc | s |
| 902 | gcggcgttcggcgcgcgccc | s |
| 903 | gcgacgtgcggcgcgcgccc | s |
| 904 | tcgtcgctgtctccg | S |
| 905 | tgtgggggttttggttttgg | S |
| 906 | aggggaggggaggggagggg | S |
| 907 | tgtgtgtgtgtgtgtgtgtgt | S |
| 908 | ctctctctctctctctctctct | sos |
| 909 | ggggtcgacgtcgagggggg | S |
| 910 | atatatatatatatatatatat | S |
| 911 | ttttttttttttttttttttttttttt | S |
| 912 | ttttttttttttttttttttt | S |
| 913 | tttttttttttttttttt | S |
| 914 | gctagaggggagggt | |
| 915 | gctagatgttagggg | |
| 916 | gcatgagggggagct | |
| 917 | atggaaggtccagggggctc | |
| 918 | atggactctggagggggctc | |
| 919 | atggaaggtccaaggggctc | |
| 920 | gagaaggggggaccttggat | |
| 921 | gagaaggggggaccttccat | |
| 922 | gagaaggggccagcactgat | |
| 923 | tccatgtggggcctgatgct | |
| 924 | tccatgaggggcctgatgct | |
| 925 | tccatgtggggcctgctgat | |
| 926 | atggactctccggggttctc | |
| 927 | atggaaggtccggggttctc | |
| 928 | atggactctggaggggtctc | |
| 929 | atggaggctccatggggctc | |
| 930 | atggactctggggggttctc | |
| 931 | tccatgtgggtggggatgct | |
| 932 | tccatgcgggtggggatgct | |
| 933 | tccatgggggtcctgatgct | |
| 934 | tccatggggtccctgatgct | |
| 935 | tccatggggtgcctgatgct | |
| 936 | tccatggggttcctgatgct | |
| 937 | tccatcgggggcctgatgct | |
| 938 | gctagagggagtgt | |
| 939 | tttttttttttttttttt | s |
| 940 | gmggtcaacgttgagggmggg | s |
| 941 | ggggagttcgttgaggggggg | s |
| 942 | tcgtcgtttccccccccccc | s |
| 943 | ttggggggttttttttttttttttt | s |
| 944 | tttaaattttaaaatttaaaata | s |
| 945 | ttggtttttttggtttttttttgg | s |
| 946 | tttcccttttccccttttcccctc | s |
| 947 | ggggtcatcgatgagggggg s | sos |
| 948 | tccatgacgttcctgacgtt | |
| 949 | tccatgacgttcctgacgtt | |
| 950 | tccatgacgttcctgacgtt | |
| 951 | tccatgacgttcctgacgtt | |
| 952 | tccatgacgttcctgacgtt | |
| 953 | tccatgacgttcctgacgtt | |
| 954 | tccatgacgttcctgacgtt | |
| 955 | tccatgacgttcctgacgtt | |
| 956 | tccatgacgttcctgacgtt | |
| 957 | tccatgacgttcctgacgtt | |
| 958 | tccatgacgttcctgacgtt | |
| 959 | gggggacgatcgtcggggg | sos |
| 960 | gggggtcgtacgacgggggg | sos |
| 961 | tttttttttttttttttttttttt | po |
| 962 | aaaaaaaaaaaaaaaaaaaaaaaa | po |
| 963 | cccccccccccccccccccccccc | po |
| 964 | tcgtcgttttgtcgttttgtcgtt | |
| 965 | tcgtcgttttgtcgttttgtcgtt | |
| 966 | tcgtcgttttgtcgttttgtcgtt | |
| 967 | tcgtcgttttgtcgttttgtcgtt | |
| 968 | ggggtcaacgttgagggggg | |
| 969 | ggggtcaacgttgagggggg | |
| 970 | ggggtcaagcttgagggggg | |
| 971 | tgctgcttcccccccccccc | |
| 972 | ggggacgtcgacgtgggggg | sos |
| 973 | ggggtcgtcgacgagggggg | sos |
| 974 | ggggtcgacgtacgtcgagggggg | sos |
| 975 | ggggaccggtacoggtgggggg | sos |
| 976 | gggtcgacgtcgagggggg | sos |
| 977 | ggggtcgacgtcgaggggg | sos |
| 978 | ggggaacgttaacgttgggggg | sos |
| 979 | ggggtcaccggtgagggggg | sos |
| 980 | ggggtcgttcgaacgagggggg | sos |
| 981 | ggggacgttcgaacgtgggggg | sos |
| 982 | tcaactttga | s |
| 983 | tcaagcttga | s |
| 984 | tcacgatcgtga | s |
| 985 | tcagcatgctga | s |
| 986 | gggggagcatgctggggggg | sos |
| 987 | gggggggggggggggggggg | sos |
| 988 | gggggacgatatcgtcgggggg | sos |
| 989 | gggggacgacgtcgtcgggggg | sos |
| 990 | gggggacgagctcgtcgggggg | sos |
| 991 | gggggacgtacgtcgggggg | sos |
| 992 | tcaacgtt | |
| 993 | tccataccggtcctgatgct | |
| 994 | tccataccggtcctaccggt | s |
| 995 | gggggacgatcgttgggggg | sos |
| 996 | ggggaacgatcgtcgggggg | sos |
| 997 | ggg ggg acg atc gtc ggg ggg | sos |
| 998 | ggg gga cga tcg tcg ggg ggg | sos |
| 999 | aaa gac gtt aaa | po |
| 1000 | aaagagcttaaa | po |
| 1001 | aaagazgttaaa | po |
| 1002 | aaattcggaaaa | po |
| 1003 | gggggtcatcgatgagggggg | sos |
| 1004 | gggggtcaacgttgagggggg | sos |
| 1005 | atgtagcttaataacaaagc | po |
| 1006 | ggatcccttgagttacttct | po |
| 1007 | ccattccacttctgattacc | po |
| 1008 | tatgtattatcatgtagata | po |
| 1009 | agcctacgtattcaccctcc | po |
| 1010 | ttcctgcaactactattgta | po |
| 1011 | atagaaggccctacaccagt | po |
| 1012 | ttacaccggtctatggaggt | po |
| 1013 | ctaaccagatcaagtctagg | po |
| 1014 | cctagacttgatctggttag | po |
| 1015 | tataagcctcgtccgacatg | po |
| 1016 | catgtcggacgaggcttata | po |
| 1017 | tggtggtggggagtaagctc | po |
| 1018 | gagctactcccccaccacca | po |
| 1019 | gccttcgatcttcgttggga | po |
| 1020 | tggacttctctttgccgtct | po |
| 1021 | atgctgtagcccagcgataa | po |
| 1022 | accgaatcagcggaaagtga | po |
| 1023 | tccatgacgttcctgacgtt | |
| 1024 | ggagaaacccatgagctcatctgg | |
| 1025 | accacagaccagcaggcaga | |
| 1026 | gagcgtgaactgcgcgaaga | |
| 1027 | tcggtacccttgcagcggtt | |
| 1028 | ctggagccctagccaaggat | |
| 1029 | gcgactccatcaccagcgat | |
| 1030 | cctgaagtaagaaccagatgt | |
| 1031 | ctgtgttatctgacatacacc | |
| 1032 | aattagccttaggtgattggg | |
| 1033 | acatctggttcttacttcagg | |
| 1034 | ataagtcatattttgggaactac | |
| 1035 | cccaatcacctaaggctaatt | |
| 1036 | ggggtcgtcgacgagggggg | sos |
| 1037 | ggggtcgttcgaacgagggggg | sos |
| 1038 | ggggacgttcgaacgtgggggg | sos |
| 1039 | tcctggcggggaagt | s |
| 1040 | ggggaacgacgtcgttgggggg | sos |
| 1041 | ggggaacgtacgtcgggggg | sos |
| 1042 | ggggaacgtacgtacgttgggggg | sos |
| 1043 | ggggtcaccggtgagggggg | sos |
| 1044 | ggggtcgacgtacgtcgagggggg | sos |
| 1045 | ggggaccggtaccggtgggggg | sos |
| 1046 | gggtcgacgtcgagggggg | sos |
| 1047 | ggggtcgacgtcgagggg | sos |
| 1048 | ggggaacgttaacgttgggggg | sos |
| 1049 | ggggacgtcgacgtggggg | sos |
| 1050 | gcactcttcgaagctacagccggcagcctctgat | |
| 1051 | cggctcttccatgaggtctttgctaatcttgg | |
| 1052 | cggctcttccatgaaagtctttggacgatgtgagc | |
| 1053 | tcctgcaggttaagt | s |
| 1054 | gggggtcgttcgttgggggg | sos |
| 1055 | gggggatgattgttgggggg | sos |
| 1056 | gggggazgatzgttgggggg | sos |
| 1057 | gggggagctagcttgggggg | sos |
| 1058 | ggttcttttggtccttgtct | s |
| 1059 | ggttcttttggtcctcgtct | s |
| 1060 | ggttcttttggtccttatct | s |
| 1061 | ggttcttggtttccttgtct | s |
| 1062 | tggtcttttggtccttgtct | s |
| 1063 | ggttcaaatggtccttgtct | s |
| 1064 | gggtcttttgggccttgtct | s |
| 1065 | tccaggacttctctcaggtttttt | s |
| 1066 | tccaaaacttctctcaaatt | s |
| 1067 | tactacttttatacttttatactt | s |
| 1068 | tgtgtgtgtgtgtgtgtgtgtgtg | s |
| 1069 | ttgttgttgttgtttgttgttgttg | s |
| 1070 | ggctccggggagggaatttttgtctat | s |
| 1071 | gggacgatcgtcggggggg | sos |
| 1072 | gggtcgtcgacgaggggggg | sos |
| 1073 | ggtcgtcgacgaggggggg | sos |
| 1074 | gggtcgtcgtcgtggggggg | sos |
| 1075 | ggggacgatcgtcggggggg | sos |
| 1076 | ggggacgtcgtcgtgggggg | sos |
| 1077 | ggggtcgacgtcgacgtcgaggggggg | sos |
| 1078 | ggggaaccgcggttggggggg | sos |
| 1079 | ggggacgacgtcgtggggggg | sos |
| 1080 | tcgtcgtcgtcgtcgtggggggg | sos |
| 1081 | tcctgccggggaagt | s |
| 1082 | tcctgcaggggaagt | s |
| 1083 | tcctgaaggggaagt | s |
| 1084 | tcctggcgggcaagt | s |
| 1085 | tcctggcgggtaagt | s |
| 1086 | tcctggcgggaaagt | s |
| 1087 | tccgggcggggaagt | s |
| 1088 | tcggggcggggaagt | s |
| 1089 | tcccggcggggaagt | s |
| 1090 | gggggacgttggggg | s |
| 1091 | ggggttttttttttgggggg | sos |
| 1092 | ggggccccccccccgggggg | sos |
| 1093 | ggggttgttgttgttgggggg | sos |

Nucleic acids having modified backbones also are included in the class of nucleic acids having antiangiogenic properties. Modified backbone nucleic acids include those having phosphorothioate, methylphosphonate, methylphosphorothioate, p-ethoxy and/or phosphorodithioate internucleotide or internucleotide bonds. Chimeric oligonucleotides having mixtures of modified and/or unmodified backbones also are included in the invention.

In the case when an antiangiogenic nucleic acid is administered in conjunction with a nucleic acid vector, it is preferred that the backbone of the antiangiogenic nucleic acid be a chimeric combination of phosphodiester and phosphorothioate bonds (or other modification of the internucleotide bonds). This is because the uptake of the plasmid vector by the cell may be hindered by the presence of completely phosphorothioate oligonucleotide. Thus when both a vector and an oligonucleotide are delivered to a subject, it is preferred that the oligonucleotide have chimeric or phosphorothioate internucleotide bonds and that the plasmid be associated with a vehicle that delivers it directly into the cell, thus avoiding the need for cellular uptake. Such vehicles are known in the art and include, for example, liposomes, electroporation devices and gene guns.

For use in the instant invention, the antiangiogenic nucleic acids can be synthesized de novo using any of a number of procedures well known in the art. Such compounds are referred to as “synthetic nucleic acids.” For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986, Garegg et al, Tet. Let. 27:4055-4058, 1986, Gaffney et al., Tet. Let. 29:2619-2622, 1988). These chemistries can be performed by a variety of automated oligonucleotide synthesizers available in the market.

Alternatively, nucleic acids can be produced on a large scale in plasmids, (see, e.g., Sambrook, et al., Molecular Cloning: A Laboratory Manual, Cold Spring Harbor Laboratory Press, New York, 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acids. The term “antiangiogenic nucleic acid” encompasses both synthetic and isolated antiangiogenic nucleic acids.

For use in vivo, nucleic acids are preferably relatively resistant to degradation (e.g., are stabilized). A “stabilized nucleic acid molecule” as used herein means a nucleic acid molecule that is relatively resistant to in vivo degradation (e.g. via an exo- or endo-nuclease). Stabilization can be a function of length or secondary structure. Antiangiogenic nucleic acids that are tens to hundreds of kilobases long are relatively resistant to in vivo degradation. For shorter antiangiogenic nucleic acids, secondary structure can stabilize and increase their effect. For example, if the 3′ end of a nucleic acid is self-complementary to an upstream region of the same nucleic acid, so that it can fold back and form a stem/loop structure by internal self-hybridization, then the nucleic acid may be stabilized and therefore may exhibit more in vivo activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the antiangiogenic nucleic acids when administered in vivo. One type of modified backbone is a phosphate backbone modification. For example, antiangiogenic nucleic acids including at least two phosphorothioate linkages at the 5′ end of the oligonucleotide and multiple phosphorothioate linkages at the 3′ end, preferably 5 or more, can in some circumstances protect the nucleic acid from degradation by intracellular exo- and endo-nucleases and thereby provide maximal activity. Other phosphate modified nucleic acids include phosphodiester modified nucleic acids, combinations of phosphodiester and phosphorothioate nucleic acids, methylphosphonate, methylphosphorothioate, phosphorodithioate, p-ethoxy and combinations thereof. Some of these combinations in CpG nucleic acids and their particular effects on immune cells is discussed in more detail in PCT Published Patent Applications PCT/US95/01570 and PCT/US97/19791, the entire contents of which are hereby incorporated by reference. Although not intending to be bound by any particular theory, it is believed that these modified nucleic acids may have increased activity relative to unmodified nucleic acids due to enhanced nuclease resistance, increased cellular uptake, increased protein binding, and/or altered intracellular localization.

Modified backbone nucleic acids, such as those having phosphorothioates bonds may be synthesized using automated techniques employing, for example, phosphoramidate or H-phosphonate chemistries. Aryl-and alkyl-phosphonates can be made, e.g., as described in U.S. Pat. No. 4,469,863. Alkylphosphotriesters, in which the charged oxygen moiety is alkylated as described in U.S. Pat. No. 5,023,243 and European Patent No. 092,574, can be prepared by automated solid phase synthesis using commercially available reagents. Methods for making other nucleic acid backbone modifications and substitutions have been described (Uhlmann, E. and Peyman, A., Chem. Rev. 90:544, 1990; Goodchild, J., Bioconjugate Chem. 1:165, 1990).

Another type of modified backbone, useful according to the invention, is a peptide nucleic acid. The backbone is composed of aminoethylglycine and supports bases which provide the nucleic acid character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-O—Me). These types of substitutions are described extensively in the literature and in particular with respect to their immunostimulating properties in Zhao et al., Bioorganic and Medicinal Chemistry Letters, 1999, 9:24:3453. Zhao et al. describes methods of preparing 2′-O—Me modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

Other stabilized nucleic acids include non-ionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The antiangiogenic nucleic acids having backbone modifications useful according to the invention in some embodiments are S- or R-chiral antiangiogenic nucleic acids. An “S chiral antiangiogenic nucleic acid” as used herein is an antiangiogenic nucleic acid wherein at least two nucleotides have a backbone modification forming a chiral center and wherein a plurality of the chiral centers have S chirality. An “R chiral antiangiogenic nucleic acid” as used herein is an antiangiogenic nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, p-ethoxy, 2′-O—Me and combinations thereof.

The chiral antiangiogenic nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein refers to an amount greater than 50%. Thus, less than all of the chiral centers may have S or R chirality as long as a plurality of the chiral centers have S or R chirality. In some embodiments at least 55%, 60%, 65%, 70%, 75%, 80%, 85%, 90%, 95%, or 100% of the chiral centers have S or R chirality. In other embodiments at least 55%, 60%, 65%, 70%, 75%, 80%, 85%, 90%, 95%, or 100% of the nucleotides have backbone modifications.

The S- and R- chiral antiangiogenic nucleic acids may be prepared by any method known in the art for producing chirally pure oligonucleotides. Stec et al teach methods for producing stereopure phosphorothioate oligodeoxynucleotides using an oxathiaphospholane. (Stec, W. J., et al., 1995, J. Am. Chem. Soc., 117:12019). Other methods for making chirally pure oligonucleotides have been described by companies such as ISIS Pharmaceuticals. US Patents which disclose methods for generating stereopure oligonucleotides include U.S. Pat. Nos. 5,883,237; 5,837,856; 5,599,797; 5,512,668; 5,856,465; 5,359,052; 5,506,212; 5,521,302; and 5,212,295, each of which is hereby incorporated by reference in its entirety.

As used herein, administration of an antiangiogenic nucleic acid is intended to embrace the administration of one or more antiangiogenic nucleic acids which may or may not differ in terms of their profile, sequence, backbone modifications and biological effect. As an example, CpG nucleic acids and T-rich nucleic acids may be administered to a single subject along with other antiangiogenic medicament(s), such as endostatin or angiostatin. In another example, a plurality of CpG nucleic acids which differ in nucleotide sequence may also be administered to a subject.

The invention encompasses the administration of the antiangiogenic nucleic acids along with other medicaments in order to provide a synergistic effect useful in the prevention and/or treatment of conditions that involve unwanted angiogenesis, such as cancer. Accordingly, methods for inhibition of angiogenesis are provided. The methods include the administration of at least one antiangiogenic nucleic acid formulated for administration to a subject. Non-nucleic acid antiangiogenesis molecules also can be administered to the subject, including, but not limited to endogenous angiogenesis inhibitors including PD 174073 and PD 166285 (Parke-Davis), SU5416 and SU6668 (Sugen), ZD 4190 and ZD 6474 (Zeneca), PTK 787 (also known as CGP79787 or ZK22584) (Novartis), Anti-VEGF mAb (Genentech), Anti-KDR mAb (ImClone), RPI 4610 (Ribozyme), TNP 470 (Abbott/TAP), AG 3340 (Agouron), Marimastat (British Biotech), Bay 12-9566 (Bayer), Neovastat (Aeterna), BMS 275291 (Bristol Myers-Squibb), CGS 27023A (Novartis), D1927 Chiroscience), D2163 (Chiroscience), Isoquinolines (Pfizer), Vitaxin (IXSYS), S-137 (Searle), S-836 (Searle), SM256 (Dupont), SG545 (Dupont), Angiostatin (EntreMed), Endostatin (EntreMed), Thalidomide (EntreMed), Squalamine (Magainin), CAI (National Cancer Institute), CM-101 (CarboMed), U-995 (Gwo-Chyang GMP), Combretastatin A-4 (Oxigene), platelet factor-4, vasostatin, thrombospondin, tissue inhibitors of metalloproteinases (TIMPs), STI412 (Sun and McMahon, Drug Discov. Today 5(8):344-353, 2000; Klohs and Hamby, Curr. Opin. Biotechnol. 10:544-549, 1999), fumagillin, non-glucocorticoid steroids and heparin and heparin fragments and antibodies to oen or more angiogenic peptides such as α-FGF, β-FGF, VEGF, IL-8, and GM-CSF. Some of the foregoing may be administered in the form of nucleic acids encoding proteins; in each case the active agent is a protein and not the nucleic acid encoding the protein.

The antiangiogenic nucleic acid molecules of the invention can be administered concurrently with, or sequentially with, the non-nucleic acid antiangiogenesis molecules described above. Coadministration may be in the form of administration of a composition containing both kinds of antiangiogenic agents, or a plurality of compositions, each of which may contain one or more than one of the antiangiogenic agents.

The invention may be used in the treatment of cancer, but is not so limited. In these methods, an effective amount of at least one antiangiogenic nucleic acid is administered to a subject having cancer, or in other instances a subject at risk of developing cancer. Other non-nucleic acid antiangiogenesis molecules also can be administered, as described above. In addition, in certain embodiments of the invention, anticancer molecules are administered in combination with the antiangiogenesis molecules.

The compounds useful in the invention may be delivered in a mixture with anti-proliferative agents (particularly anticancer agents) which are not antiangiogenic nucleic acids. One of ordinary skill in the art is familiar with a variety of anti-proliferative agents which are used in the medical arts to treat proliferative diseases such as cancer. These anti-cancer agents may act by directly killing cells, such as cancer cells (i.e., direct action anti-cancer agents), or alternatively they may act by sensitizing cells to direct action anti-cancer agents (i.e., indirect action anti-cancer agents). Those of skill in the art will recognize the distinction and are familiar with agents of either class. Anticancer agents include, but are not limited to, the following sub-classes of compounds:

Antineoplastic agents such as: Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198 ; Hydroxyurea; Idarubicin Hydrochloride; Ifosfamide; Ilmofosine; Interferon Alfa-2a; Interferon Alfa-2b ; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safinol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones including desoxyepothilones (A, R═H; B, R═Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

Antiproliferative agent: Piritrexim Isethionate.

Radioactive agents: Fibrinogen I 125; Fludeoxyglucose F 18 ; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125 Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

The present invention further includes nucleic acid molecules formulated into a pharmaceutical composition for the inhibition of angiogenesis. The pharmaceutical compositions of the invention include those suitable for oral, rectal, nasal, topical (including buccal and sublingual), vaginal or parenteral (including subcutaneous, intramuscular, intravenous, intratumoral and intradermal) administration.

The nucleic acids are delivered in effective amounts. In general, the term “effective amount” of a nucleic acid refers to the amount necessary or sufficient to realize a desired biologic effect. Specifically, the effective amount is that amount that reduces the rate or inhibits altogether angiogenesis. For instance, when the subject bears a tumor having a blood supply, an effective amount is that amount which decreases or eliminates all together the blood supply to the tumor. Additionally, an effective amount may be that amount which prevents an increase or causes a decrease in new blood vessels, e.g., those vessels supplying a tumor. The effective amount may vary depending upon whether the antiangiogenic nucleic acid is used alone or in combination with other therapeutics, or in single or multiple dosages. In some instances, it is envisioned that the combination of antiangiogenic nucleic acids with other therapeutic agents (which are themselves not antiangiogenic nucleic acids) can result in a synergism between the two compound classes, and thereby would require less of one or both compounds in order to observe the desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. As mentioned above, the effective amount for any particular application can vary depending on such factors as the type of condition having unwanted angiogenesis being treated or prevented, the particular nucleic acid being administered (e.g. the number of unmethylated CpG motifs or their location in the nucleic acid), the use of another antiangiogenesis agent, the size of the subject, or the severity of the disease or condition. One of ordinary skill in the art can empirically determine the effective amount of a particular nucleic acid molecule without necessitating undue experimentation.

Subject doses of the compounds described herein typically range from about 0.1 μg to 10 mg per administration, which depending on the application could be given hourly, daily, weekly, or monthly and any other amount of time therebetween. More typically doses range from about 10 μg to 5 mg per administration, and most typically from about 100 μg to 1 mg, with 2-4 administrations being spaced hours, days or weeks apart. In some embodiments, however, parenteral doses for these purposes may be used in a range of 5 to 10,000 times higher than the typical doses described above.

For any compound described herein the therapeutically effective amount can be initially determined from animal models, e.g. the animal models described herein or those well known in the art. A therapeutically effective dose can also be determined from human data for CpG nucleic acids which have been tested in humans (human clinical trials have been initiated and the results publicly disseminated) and for compounds which are known to exhibit similar pharmacological activities, such as other antiangiogenesis agents. Higher doses may be required for parenteral administration, as described above. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the nucleic acid can be administered to a subject by any mode that delivers the nucleic acid to a subject. “Administering” the pharmaceutical composition of the present invention may be accomplished by any means known to the skilled artisan. Some routes of administration include but are not limited to oral, intranasal, intratracheal, inhalation, ocular, vaginal, rectal, parenteral (e.g. intramuscular, intradermal, intravenous, intratumoral or subcutaneous injection) and direct injection.

For oral administration, the compounds (i.e., antiangiogenic nucleic acid molecules and optionally other antiangiogenesis agents) can be delivered alone without any pharmaceutical carriers or formulated readily by combining the active compound(s) with pharmaceutically acceptable carriers well known in the art. The term “pharmaceutically-acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions may also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

The nucleic acid molecules and/or agents (e.g., antiangiogenesis agents, anticancer agents) may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The nucleic acids or other therapeutics useful in the invention may be delivered in mixtures with additional antiangiogenesis agent(s). A mixture may consist of several antiangiogenesis agents in addition to the nucleic acid.

A variety of administration routes are available. The particular mode selected will depend, of course, upon the particular nucleic acid molecules or other agents selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. In still other embodiments, the agents and nucleic acids are formulated with GELFOAM, a commercial product consisting of modified collagen fibers that degrade slowly.

The nucleic acid may be directly administered to the subject or may be administered in conjunction with a pharmaceutically acceptable carrier or a delivery vehicle. The nucleic acid and optionally other therapeutic agents may be administered alone (e.g. in saline or buffer) or using any delivery vehicles known in the art. One type of delivery vehicle is referred to herein as a nucleic acid delivery complex. A “nucleic acid delivery complex” shall mean a nucleic acid molecule associated with (e.g. ionically or covalently bound to; or encapsulated within) a targeting means (e.g. a molecule that results in higher affinity binding to target cell (e.g. dendritic cell surfaces and/or increased cellular uptake by target cells). Examples of nucleic acid delivery complexes include nucleic acids associated with: a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to reduce significant uncoupling prior to internalization by the target cell. However, the complex may be cleavable under appropriate conditions within the cell so that the nucleic acid may be released in a functional form.

The nucleic acid molecules may be delivered by non-invasive methods as described above. Non-invasive delivery of compounds is desirable for treatment of children, elderly, animals, and even adults and also to avoid the risk of needle-stick injury. Delivery vehicles for delivering compounds to mucosal surfaces have been described and include but are not limited to: cochleates, emulsomes, ISCOMs, liposomes, live bacterial vectors (e.g., Salmonella, Escherichia coli, Bacillus calmatte-guerin, Shigella, Lactobacillus), live viral vectors (e.g., Vaccinia, adenovirus, Herpes Simplex), microspheres, nucleic acid vaccines, polymers (e.g. carboxymethylcellulose, chitosan), polymer rings, proteosomes, sodium fluoride, transgenic plants, virosomes, and virus-like particles.

### EXAMPLES

1. Background

1.1. Angiogenesis

Angiogenesis describes the active biological process of blood vessel formation from pre-existing microvasculature (1, 2). In multi-celled organisms this is a highly organized and tightly regulated process that occurs normally during development, inflammation, and tissue repair. The importance of angiogenesis is reflected in the need of mammalian cells for oxygen and nutrients. Mammalian cells must be within a 200 μM distance of blood vessels, which is the diffusion limit for oxygen (3). Thus the overall driving factor for angiogenesis is the requirement for oxygen and nutrients. The normal regulation of angiogenesis is mediated by the balance between pro- and anti-angiogenic factors that are released in the tissues and are influenced by local environmental factors.

1.2. Angiogenesis and Neoplasms

In a neoplastic situation, the balances of these pro- and anti-angiogenic factors are generally skewed in favor of angiogenesis. In this setting, angiogenesis is generally a highly disorganized and loosely regulated process that is an absolute requirement for the continued growth of neoplasms (3). Further, there is a direct correlation between the extent of vascularization found in neoplasms and the potential for metastasis (4).

1.3. Angiogenesis and Chemokines

There are a number of pro- and anti-angiogenic factors that have been described to date (3). The focus of this analysis will be on the chemokines interferon-γ-inducible protein (IP-10) and monokine induced by interferon-γ (MIG). Chemokines are a collection of cytokines that possess chemoattracting properties (for review see (5)). Chemokines are classified on the basis of the motif displayed by the first two cysteine residues present in the protein (CXC, CC, C, or CX3C), and they signal through G-protein coupled, seven-transmembrane receptors. Initially identified for their influence on hemopoietic cell migration, chemokines are now known to influence a number of physiological and pathological process including angiogenesis and angiostasis (5).

IP-10 and MIG belong to a subset of the family of CXC chemokines (2) that bind the chemokine receptor CXCR3 (6). The CXC chemokine family can be further subdivided based on the presence or absence of a Glu-Leu-Arg or ELR motif at the NH2 terminus of the chemokine. CXC chemokines that contain the ELR motif are potent promoters of angiogenesis whereas CXC chemokines that lack the ELR motif, as is the case for IP-10 and MIG, are potent inhibitors of angiogenesis (2).

2. Material and Methods

2.1. ODNs

ODN 1826 (TCCATGACGTTCCTGACGTT; SEQ ID NO: 69)

2.2 Matrigel®-(BD)

Matrix solution is liquid at 4° C. and solidifies at room temperature. When injected in vivo Matrigel solidifies to form a plug. Matrigel allows for the delivery of angiogenic promoters such as basic fibroblastic growth factor (bFGF) for the induction of angiogenesis. Plugs can then be removed to evaluate the level of angiogenesis as identified by the concentration of hemoglobin present. This system can be used to evaluate the anti-angiogenic potential of different compounds.

2.3 Hemoglobin Quantification Kit

Drabkin method reagent kit (Sigma)

2.4 Protein Quantification

Protein quantification kit (BioRad)

2.5 Experimental Design

For each group of 5 mice, the Matrigel was prepared as follows:

Group 1—Matrigel alone

3.5 mL of Matrigel

500 μL/mouse was injected subcutaneously (SC) right of center of the abdomen

Group 2—Matrigel+bFGF (150 ng/mL)+heparin (40 units/mL)

52.5 μL bFGF (10 μg/mL)

23.2 μL heparin (6039 units/mL)

3.42 mL Matrigel

500 μL/mouse was injected SC right of center of the abdomen

Group 3—Matrigel+bFGF (150 ng/mL)+heparin (40 units/mL)+oligo 1826 (1 mg/mL)

52.5 μL bFGF (10 μg/mL)

23.2 μL heparin (6039 units/mL)

233 μL oligo 1826 (15 mg/mL)

3.19 mL Matrigel

500 μL/mouse was injected SC right of center of the abdomen

Group 4—Matrigel+bFGF (150 ng/mL)+heparin (40 units/mL)

52.5 μL bFGF (10 μg/mL)

23.2 μL heparin (6039 units/mL)

3.42 mL Matrigel

500 μL/mouse was injected SC right of center of the abdomen

This group received daily SC injections, for 6 days, of 100 μL of ODN 1826 (1 mg/mL) on the opposite flank from the Matrigel plug.

2.6 Determination of Hemoglobin and Total Protein Content of Matrigel Plugs

On day 6 the animals were euthanised and the Matrigel plugs collected. The plugs were placed in 0.3 mL of sterile PBS and placed at 4° C. over night to allow the Matrigel to liquify. The hemoglobin and total protein content of the Matrigel plugs was determined using the methods described above. The hemoglobin content of the Matrigel plugs was expressed as (mg/mL)/mg of total protein.

3. Preliminary Results

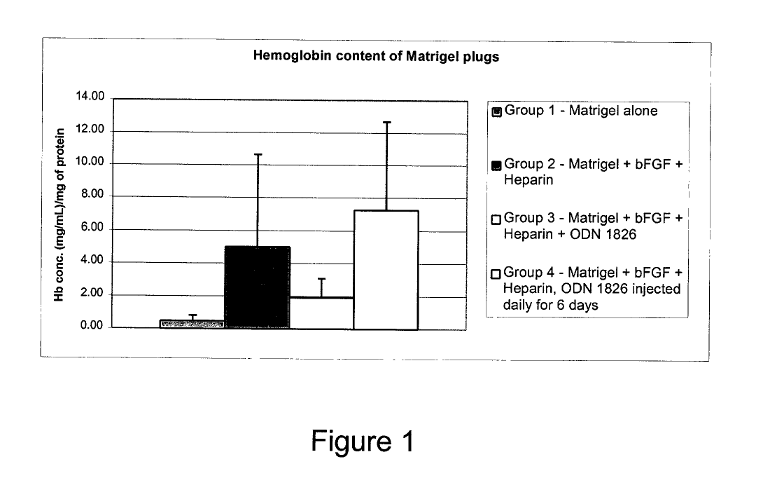

When angiogenic factors were added to the Matrigel (Group 2), there was a significant increase in the amount of hemoglobin present in the Matrigel plug at 6 days when compared to Matrigel alone (Group 1) (p<0.05). (See FIG. 1.)

When CpG was included in the Matrigel plug along with the angiogenic factors (Group 3), there was a greater than 2 fold decrease in the amount of hemoglobin present in the Matrigel plug at 6 days when compared to the Matrigel containing the angiogenic factors (Group 2). (See FIG. 1.)

When CpG was administered daily by subcutaneous injection, rather than present in the Matrigel plug, to the mouse in the flank opposite to the Matrigel plug which contained angiogenic factors (Group 4) there was no significant difference in the amount of hemoglobin present in the Matrigel plug at 6 days when compared to Matrigel containing the angiogenic factors (Group 2). (See FIG. 1.)

These preliminary results suggest that the inclusion of CpG ODN directly within the Matrigel (Group 3) had a negative influence on angiogenesis. Although daily delivery of CpG to the opposite flank from the Matrigel plug did not appear to influence angiogenesis, it is possible that CpG administered intravenously or subcutaneously in a region closer to the plug (and accordingly tumor mass) would manifest anti-angiogenic activity. CpG ODN may have to be present in the vicinity of active angiogenesis in order to have a negative influence.

4. References

1. Carneliet, P. 2000. Mechanisms of angiogenesis and arteriogenesis. Nat Med. 6: 389-95.

2. Belperio, J. A., et al. 2000. CXC chemokines in angiogenesis. J Leukoc Biol. 68: 1-8.

3. Carmeliet, P., R. K. Jain. 2000. Angiogenesis in cancer and other diseases. Nature. 407: 249-57.

4. Zetter, B. R. 1998. Angiogenesis and tumor metastasis. Annu Rev Med. 49: 407-24.

5. Rossi, D., A. Zlotnik. 2000. The biology of chemokines and their receptors. Annu Rev Immunol. 18: 217-42.

6. Loetscher, M., et al. 1996. Chemokine receptor specific for IP10 and MIG: structure, function, and expression in activated T-lymphocytes. J Exp Med. 184: 963-9.

7. Coughlin, C. M., et al. 1998. Tumor cell responses to IFNgamma affect tumorigenicity and response to IL-12 therapy and antiangiogenesis. Immunity. 9: 25-34.

8. Strasly, M., et al. 2001. IL-12 inhibition of endothelial cell functions and angiogenesis depends on lymphocyte-endothelial cell cross-talk. J Immunol. 166: 3890-9.

9. Kanegane, C., et al. 1998. Contribution of the CXC chemokines IP-10 and Mig to the antitumor effects of IL-12. J Leukoc Biol. 64: 384-92.

Equivalents

It should be understood that the preceding is merely a detailed description of certain preferred embodiments. It therefore should be apparent to those of ordinary skill in the art that various modifications and equivalents can be made without departing from the spirit and scope of the invention. It is intended that the invention encompass all such modifications within the scope of the appended claims. All references, patents and patent applications and publications that are cited or referred to in this application are incorporated in their entirety herein by reference.

I claim:

1. A method of inhibiting angiogenesis in a subject in need of such treatment comprising administering to the subject at least one antiangiogenic nucleic acid molecule in an amount effective to inhibit angiogenesis in the subject.

2. The method of claim 1, wherein the at least one antiangiogenic nucleic acid molecule comprises at least one sequence set forth as SEQ ID NOs: 1-1093.

3. The method of claim 1, wherein two or more antiangiogenic nucleic acid molecules are administered.

4. The method of claim 1, further comprising administering to the subject at least one non-nucleic acid angiogenesis inhibitor molecule.

5. The method of claim 1, wherein the angiogenesis is associated with a condition selected from the group consisting of a solid tumor growth, a tumor metastasis, and a precancerous lesion.

6. The method of claim 1, wherein the nucleic acid is a CpG nucleic acid having an unmethylated CpG motif.

7. The method of claim 1, wherein the nucleic acid is a T-rich nucleic acid.

8. The method of claim 1, wherein the nucleic acid is a poly G nucleic acid.

9. The method of claim 1, wherein the nucleic acid is isolated.

10. The method of claim 1, wherein the nucleic acid does not encode a protein having antiangiogenesis activity.

11. The method of claim 1, wherein the nucleic acid has a modified backbone.

12. The method of claim 11, wherein the modified backbone is a phosphate backbone modification.

13. The method of claim 11, wherein the modified backbone is a peptide modified oligonucleotide backbone.

14. The method of claim 1, further comprising administering to the subject at least one anticancer agent.

15. The method of claim 1, further comprising administering to the subject at least one antiarthritis agent.

16. The method of claim 6, wherein the CpG nucleic acid comprises:
5′X₁X₂CGX₃X₄3′
wherein C is unmethylated, and wherein X₁X₂ and X₃X₄ are nucleotides.

17. The method of claim 16, wherein the 5′ X₁ X₂CGX₃ X₄3′ sequence is a non-palindromic sequence.

18. The method of claim 16, wherein the CpG nucleic acid has 8 to 100 nucleotides.

19. The method of claim 16, wherein X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA.

20. The method of claim 16, wherein X₁X₂ are selected from the group consisting of GpA and GpT and X₃X₄ are TpT.

21. The method of claim 16, wherein X₁X₂ are both purines and X₃X₄ are both pyrimidines.

22. The method of claim 16, wherein X₂ is a T and X₃ is a pyrimidine.

23. The method of claim 16, wherein the CpG nucleic acid is 8 to 40 nucleotides in length.

24. The method of claim 16, wherein the CpG nucleic acid has a sequence selected from the group consisting of SEQ ID NOs: 1, 3, 4, 14-16, 18-24, 28, 29, 33-46, 49, 50, 52-56, 58, 64-67, 69, 71, 72, 76-87, 90, 91, 93, 94, 96, 98, 102-124, 126-128, 131-133, 136-141, 146-150, 152-153, 155-171, 173-178, 180-186, 188-198, 201, 203-214, 216-220, 223, 224, 227-240, 242-256, 258, 260-265, 270-273, 275, 277-281, 286-287, 292, 295-296, 300, 302, 305-307, 309-312, 314-317, 320-327, 329, 335, 337-341, 343-352, 354, 357, 361-365, 367-369, 373-376, 378-385, 388-392, 394, 395, 399, 401-404, 406-426, 429-433, 434-437, 439, 441-443, 445, 447, 448, 450, 453-456, 460-464, 466-469, 472-475, 477, 478, 480, 483-485, 488, 489, 492, 493, 495-502, 504-505, 507-509, 511, 513-529, 532-541, 543-555, 564-566, 568-576, 578, 580, 599, 601-605, 607-611, 613-615, 617, 619-622, 625-646, 648-650, 653-664, 666-697, 699-706, 708, 709, 711-716, 718-732, 736, 737, 739-744, 746, 747, 749-761, 763, 766-767, 769, 772-779, 781-783, 785-786, 7900792, 798-799, 804-808, 810, 815, 817, 818, 820-832, 835-846, 849-850, 855-859, 862, 865, 872, 874-877, 879-881, 883-885, 888-904, and 909-913.

25. The method of claim 7, wherein the T-rich nucleic acid is a poly T nucleic acid comprising
5′TTTT3′.

26. The method of claim 25, wherein the poly T nucleic acid comprises
5′X₁X₂TTTTX₃X₄3′
wherein X₁, X₂, X₃ and X₄ are nucleotides.

27. The method of claim 25, wherein the T rich nucleic acid comprises a plurality of poly T nucleic acid motifs.

28. The method of claim 26, wherein X₁X₂ is TT.

29. The method of claim 26, wherein X₃X₄ is TT.

30. The method of claim 26, wherein X₁X₂ is selected from the group consisting of TA, TG, TC, AT, AA, AG, AC, CT, CC, CA, CG, GT, GG, GA, and GC.

31. The method of claim 26, wherein X₃X₄ is selected from the group consisting of TA, TG, TC, AT, AA, AG, AC, CT, CC, CA, CG, GT, GG, GA, and GC.

32. The method of claim 25, wherein the T rich nucleic acid comprises a nucleotide composition of greater than 25% T.

33. The method of claim 7, wherein the T rich nucleic acid comprises a nucleotide composition of greater than 25% T.

34. The method of claim 33, wherein the T rich nucleic acid comprises a nucleotide composition of greater than 30% T.

35. The method of claim 33, wherein the T rich nucleic acid comprises a nucleotide composition of greater than 50% T.

36. The method of claim 33, wherein the T rich nucleic acid comprises a nucleotide composition of greater than 60% T.

37. The method of claim 33, wherein the T rich nucleic acid comprises a nucleotide composition of greater than 80% T.

38. The method of claim 7, wherein the T rich nucleic acid comprises at least 20 nucleotides.

39. The method of claim 7, wherein the T rich nucleic acid comprises at least 24 nucleotides.

40. The method of claim 8, wherein the poly G nucleic acid comprises:
5′X₁X₂GGGX₃X₄3′
wherein X₁, X₂, X₃, and X₄ are nucleotides.

41. The method of claim 40, wherein at least one of X₃ and X₄ are a G.

42. The method of claim 40, wherein both of X₃ and X₄ are a G.

43. The method of claim 8, wherein the poly G nucleic acid comprises the following formula:
5′GGGNGGG3′
wherein N represents between 0 and 20 nucleotides.

44. The method of claim 8, wherein the poly G nucleic acid comprises the following formula:
5′GGGNGGGNGGG3′
wherein N represents between 0 and 20 nucleotides.

45. The method of claim 8, wherein the poly G nucleic acid is free of unmethylated CG dinucleotides

46. The method of claim 45, wherein the poly G nucleic acid is selected from the group consisting of SEQ ID NOs: 5, 6, 73, 215, 267-269, 276, 282, 288, 297-299, 355, 359, 386, 387, 444, 476, 531, 557-559, 733, 768, 795, 796, 914-925, 928-931, 933-936, and 938.

47. The method of claim 8, wherein the poly G nucleic acid includes at least one unmethylated CG dinucleotide.

48. The method of claim 47, wherein the poly G nucleic acid is selected from the group consisting of SEQ ID NOs: 67, 80-82, 141, 147, 148, 173, 178, 183, 185, 214, 224, 264, 265, 315, 329, 434, 435, 475, 519, 521-524, 526, 527, 535, 554, 565, 609, 628, 660, 661, 662, 725, 767, 825, 856, 857, 876, 892, 909, 926, 927, 932, and 937.

49. The method of claim 1, wherein the nucleic acid is a synthetic nucleic acid.

50. The method of claim 9, wherein the nucleic acid is administered on a routine schedule.

51. The method of claim 1, wherein the angiogenesis is associated with a condition selected from the group consisting of rheumatoid arthritis, psoriasis, diabetic retinopathy, retinopathy of prematurity, macular degeneration, corneal graft rejection, neovascular glaucoma, retrolental fibroplasia, rubeosis, Osler-Webber Syndrome, myocardial angiogenesis, plaque neovascularization, telangiectasia, hemophiliac joints, angiofibroma, wound granulation, intestinal adhesions, atherosclerosis, scleroderma, and hypertrophic scars.

52. The method of claim 1, wherein the nucleic acid is not an antisense molecule.

53. A pharmaceutical composition comprising an amount of at least one antiangiogenic nucleic acid molecule effective to inhibit angiogenesis and a pharmaceutically acceptable carrier.

54. The pharmaceutical composition of claim 53, wherein the at least one antiangiogenic nucleic acid molecule comprises at least one sequence set forth as SEQ ID NOs: 1-1093.

55. The pharmaceutical composition of claim 53, wherein two or more antiangiogenic nucleic acid molecules are administered.

56. The pharmaceutical composition of claim 53, further comprising at least one non-nucleic acid angiogenesis inhibitor molecule.

57. The pharmaceutical composition of claim 53, wherein the antiangiogenic nucleic acid molecule has a modified backbone.

58. The pharmaceutical composition of claim 57, wherein the modified backbone is a phosphate modified backbone.

59. The pharmaceutical composition of claim 58, wherein the phosphate modified backbone is a phosphorothioate modified backbone.

60. The pharmaceutical composition of claim 53, further comprising an anticancer agent.

61. The pharmaceutical composition of claim 53, wherein the nucleic acid is a CpG nucleic acid.

62. The pharmaceutical composition of claim 53, wherein the nucleic acid is a T-rich nucleic acid.

63. The pharmaceutical composition of claim 53, wherein the nucleic acid is a poly G nucleic acid.

64. The pharmaceutical composition of claim 53, wherein the nucleic acid is isolated.

65. The pharmaceutical composition of claim 53, wherein the nucleic acid is not an antisense molecule.

66. A kit comprising
a first container housing at least one antiangiogenic nucleic acid molecule, and
instructions for administering the antiangiogenic nucleic acid to a subject having a condition characterized by unwanted angiogenesis.

67. The kit of claim 66, wherein the antiangiogenic nucleic acid has a modified backbone.

68. The kit of claim 67, wherein the modified backbone is a phosphate modified backbone.

69. The kit of claim 67, wherein the phosphate modified backbone is a phosphorothioate modified backbone.

70. The kit of claim 65, further comprising a second container housing at least one non-nucleic acid antiangiogenic agent.

71. The kit of claim 65, further comprising a second container housing at least one anticancer agent.

72. The kit of claim 69, further comprising a third container housing at least one anticancer agent.

73. The kit of claim 65, wherein the nucleic acid is not an antisense molecule.

74. The kit of claim 65, wherein the instructions relate to administering the antiangiogenic nucleic acid to a subject having a condition selected from the group consisting of rheumatoid arthritis, psoriasis, diabetic retinopathy, retinopathy of prematurity, macular degeneration, corneal graft rejection, neovascular glaucoma, retrolental fibroplasia, rubeosis, Osler-Webber Syndrome, myocardial angiogenesis, plaque neovascularization, telangiectasia, hemophiliac joints, angiofibroma, wound granulation, intestinal adhesions, atherosclerosis, scleroderma, and hypertrophic scars.
